(19) United States
(12) Patent Application Publication
(10) Pub. No.: US 2010/0010294 A1
(43) Pub. Date: Jan. 14, 2010

(54) TEMPORARILY POSITIONABLE MEDICAL DEVICES

(75) Inventors: Sean P. Conlon, Loverland, OH (US); Robert M. Trusty, Cincinnati, OH (US)

Correspondence Address: K&L GATES LLP, 535 SMITHFIELD STREET, PITTSBURGH, PA, 15222, US

(73) Assignee: Ethicon Endo-Surgery, Inc., Cincinnati, OH (US)

(21) Appl. No.: 12/170,862

(22) Filed: Jul. 10, 2008

(51) Int. Cl. A61B 1/00; A61B 17/00

(52) U.S. Cl. 600/104; 606/1

(57) ABSTRACT

A temporarily positionable device includes a temporarily positionable body and an attachment mechanism formed integral with the temporarily positionable body. The attachment mechanism is to attach to body tissue. The attachment mechanism includes a fastener integral with the temporarily positionable body to attach the temporarily positionable body to the body tissue. At least one fastener has a deployed position and an undeployed position. An applier is used to move the fastener from the undeployed position to the deployed position. The temporarily positionable device can be disposed at a first location adjacent to body tissue. The temporarily positionable device can be attached to the body tissue at the first location fastener by simultaneously moving the fastener from the undeployed position to the deployed position.

### BACKGROUND

The various embodiments relate generally to temporarily positionable medical devices. More particularly, the various embodiments are directed to temporarily positionable medical devices, appliers therefor, and attachment mechanisms for use therewith. A variety of temporarily positionable medical devices and appliers for attaching the medical devices to body tissue are disclosed.

It is desirable to introduce various temporarily positionable medical devices, appliers, and attachments mechanisms inside a patient's body using minimally invasive surgical procedures. The introduction and placement of such temporarily positionable medical devices, appliers, and attachments mechanisms inside a patient's body should be quick, easy, efficient, and reversible.

Endoscopic and laparoscopic minimally invasive procedures have been used for introducing medical devices inside a patient and for viewing portions of the patient's anatomy. To view a desired treatment region of the anatomy (e.g., worksite), a clinician (e.g., a surgeon) may insert a rigid or flexible endoscope inside the patient. The clinician also may insert surgical devices through one or more working channels of the endoscope to perform various key surgical activities (KSA). A typical image obtained with an endoscope is different than that of a typical image obtained with a laparoscope. An endoscope employs a camera to render images of the worksite and provides wider angle images. Thus, an endoscope can operate at shorter working distances than a laparoscope. Because the camera is part of the endoscope, during a procedure, the clinician is required to bring the tip of the endoscope close to the worksite. This eliminates the “stadium view” of the surgical site that is preferred and desired by many clinicians. Furthermore, the ability of the clinician to “triangulate” his actions between the camera and the surgical tools is compromised when all devices are located along a single axis. Furthermore, introducing the camera and the surgical tools through working channels of the endoscope compromises its flexibility. Also, to reach the worksite with a flexible endoscope, the clinician often must navigate the endoscope through tortuous paths and, thus, the rotational orientation of the endoscope may not be aligned with the expected surgical view of the worksite. Correcting the orientation can be very difficult when operating outside of an internal body lumen. Finally, the presence of the camera and associated wiring within the endoscope takes up valuable space that could be used for more sophisticated and/or larger therapeutic or surgical medical devices.

Accordingly, there is a need for temporarily positionable medical devices, appliers therefor, and attachment mechanisms for use therewith. There is also a need for attachment mechanisms that may be used with a variety of temporarily positionable medical devices and appliers for attaching the medical devices to internal portions of the patient's anatomy.

### FIGURES

The novel features of the various embodiments are set forth with particularity in the appended claims. The various embodiments, however, both as to organization and methods of operation may best be understood by reference to the following description, taken in conjunction with the accompanying drawings as follows.

FIG. 1 is a perspective view of one embodiment of a system for applying a temporarily positionable medical device inside a patient.

FIG. 2 is a perspective view of one embodiment of a deployment handle for use with the applier and attachment mechanism illustrated in FIG. 1.

FIG. 3 is a perspective view of one embodiment of a remote camera attached to one embodiment of a camera applier device in a pre-fired position.

FIG. 4 is an exploded view of one embodiment of the temporarily positionable medical device and applier therefor shown in FIG. 3 in a post-fired released position after being fired from the applier.

FIG. 5 is a perspective view of one embodiment of a temporarily positionable medical device.

FIG. 6 is a cross-sectional view of the temporarily positionable medical device shown in FIG. 5.

FIG. 7 is a cross-sectional view of the temporarily positionable medical device shown in FIG. 5 attached to the abdominal wall with one or more fasteners.

FIG. 8 is a perspective view of one embodiment of a forward and rearward viewing temporarily positionable medical device released from an applier showing a forward image acquisition portion.

FIG. 9 is a perspective view of one embodiment of a forward and rearward viewing temporarily positionable medical device shown in FIG. 8 released from an applier showing a rearward image acquisition portion.

FIG. 10 is a cross-sectional view of one embodiment of the forward and rearward viewing temporarily positionable medical device shown in FIGS. 8 and 9.

FIG. 11 is a cross-sectional view of one embodiment of the temporarily positionable medical device shown in FIG. 10 attached to the abdominal wall with one or more fasteners.

FIG. 12 is a perspective view of one embodiment of a temporarily positionable medical device showing a rearward image acquisition portion.

FIG. 13 is a top view of the embodiment of the temporarily positionable medical device shown in FIG. 12.

FIG. 14 is a bottom view of one embodiment of the temporarily positionable medical device shown in FIG. 12.

FIG. 15 is an exploded perspective view of one embodiment of the temporarily positionable medical device shown in FIG. 12.

FIG. 16 is a bottom perspective view of one embodiment of the temporarily positionable medical device shown in FIG. 12 with fasteners located in a retracted position.

FIG. 17 is a bottom perspective view of one embodiment of the temporarily positionable medical device shown in FIG. 12 with fasteners located in an extended, or fired, position, extending from corresponding slots.

FIG. 18 is a cross-sectional view of one embodiment of a fastener in a fully retracted state, the undeployed position, disposed completely within a slot such that a sharp tip is not exposed.

FIG. 19 is a cross-sectional view of one embodiment of a fastener rotated about half way through its range of rotation, about 90 degrees as a result of a clockwise rotation of an actuator.

FIG. 20 is a cross-sectional view of one embodiment of a fastener actuator rotated clockwise to its fullest extent, with a raised rib having been urged past the detent rib.

FIG. 21 is a cross-sectional view of one embodiment of a fastener actuator that has been advanced counterclockwise compared to the position shown in FIG. 20, and a fastener is rotated approximately halfway through its range.

FIG. 22 is a top view of one embodiment of a temporarily positionable medical device with the actuator omitted to illustrate the positions of the links when the fasteners are in the retracted position.

FIG. 23 is a top view of one embodiment of a temporarily positionable medical device with the actuator omitted to illustrate the positions of the links when the fasteners are in the extended/fired position.

FIG. 24 illustrates one embodiment of a deployment handle and applier configured to position, actuate, deactuate, remove, or reposition a temporarily positionable medical device through a flexible shaft.

FIG. 25 is an exploded perspective view of one embodiment of the deployment handle, applier, and flexible shaft shown in FIG. 24.

FIG. 26 is a side view of one embodiment of the deployment handle, applier, and flexible shaft shown in FIG. 24 with one of the two body halves omitted showing the internal components in the unapplied, non-actuated position.

FIG. 27 is a side view of one embodiment of the deployment handle, applier, and flexible shaft shown in FIG. 24 with one of the two body halves omitted showing the internal components in the applied, actuated position.

FIG. 28 is an enlarged fragmentary side view of one embodiment of a linear to rotary cam mechanism of the applier shown in FIG. 24.

FIG. 29 is an enlarged top perspective view of one embodiment of a camera shroud of the applier shown in FIG. 24.

FIG. 30 is an enlarged bottom perspective view of one embodiment of a camera shroud and actuator portion of the applier shown in FIG. 24.

FIG. 31 is a partially cutaway view end view of one embodiment of a camera shroud of the applier shown in FIG. 24.

FIG. 32 illustrates one embodiment of a temporarily positionable medical device comprising forward and rearward image acquisition capabilities.

FIG. 33 illustrates one embodiment of a temporarily positionable medical device comprising a tissue retraction clip.

FIG. 34 illustrates one embodiment of a temporarily positionable medical device comprising a tissue clamp.

FIG. 35 illustrates one embodiment of a temporarily positionable medical device comprising a stabilizer clamp.

FIG. 36 illustrates one embodiment of a temporarily positionable medical device comprising an electrical power distributor, a light source, and a camera.

FIG. 37 illustrates one embodiment of a temporarily positionable medical device comprising a tissue spreader to create space between layers of tissue.

FIG. 38 is a cross-sectional view of a stomach cavity, gastrointestinal tract, and abdominal wall showing an endoscopic trocar intubated within the stomach cavity through the gastrointestinal tract.

FIG. 39 is a cross-sectional view of the stomach cavity, gastrointestinal tract, and abdominal wall shown in FIG. 38 showing an access device extending from the distal end of the endoscopic trocar.

FIG. 40 is a cross-sectional view of the stomach cavity, gastrointestinal tract, and abdominal wall shown in FIG. 39 showing a dilating balloon inserted through an opening in the stomach wall formed by the access device.

FIG. 41 is a cross-sectional view of the stomach cavity, gastrointestinal tract, and abdominal wall shown in FIG. 40 showing a distal end of the endoscopic trocar intubated inserted through the dilated opening formed in the stomach wall.

FIG. 42 is a cross-sectional view of the stomach cavity, gastrointestinal tract, and abdominal wall shown in FIG. 41 showing the flexible shaft and the applier extended through the distal end of the endoscopic trocar.

FIG. 43 is a cross-sectional view of the stomach cavity, gastrointestinal tract, and abdominal wall shown in FIG. 42 showing one embodiment of a temporarily positionable medical device attached to the abdominal wall.

### DESCRIPTION

Before explaining the various embodiments in detail, it should be noted that the embodiments are not limited in their application or use to the details of construction and arrangement of parts illustrated in the accompanying drawings and description. The illustrative embodiments may be positioned or incorporated in other embodiments, variations and modifications, and may be practiced or carried out in various ways. For example, the temporarily positionable devices disclosed herein are illustrative only and not meant to limit the scope or application thereof. Furthermore, unless otherwise indicated, the terms and expressions employed herein have been chosen for the purpose of describing the illustrative embodiments for the convenience of the reader and are not to limit the scope thereof.

In the following description, like reference characters designate like or corresponding parts throughout the several views. Also, in the following description, it is to be understood that terms such as front, back, inside, outside, and the like are words of convenience and are not to be construed as limiting terms. Terminology used herein is not meant to be limiting insofar as devices described herein, or portions thereof, may be attached or utilized in other orientations. The various embodiments will be described in more detail with reference to the drawings.

Various embodiments of temporarily positionable devices disclosed herein may be introduced within a patient using minimally invasive surgical techniques or conventional open surgical techniques. Minimally invasive techniques provide more accurate and effective access of the worksite for diagnostic and treatment procedures. In some instances it may be advantageous to introduce the temporarily positionable devices into the patient using a combination of minimally invasive and open surgical techniques. Accordingly, various embodiments of temporarily positionable devices disclosed herein may be used in endoscopic and/or laparoscopic surgical procedures, conventional laparotomies, or any combinations thereof. In one embodiment, the temporarily positionable devices disclosed herein may be introduced through a natural opening of the body such as the mouth, anus, and/or vagina. Once the devices are introduced through a natural opening, internal organs may be reached using trans-organ or translumenal surgical procedures. In a natural orifice endoscopic translumenal procedure, the flexible portion of an endoscope is introduced into the patient through one or more natural orifices to view and treat diseased tissue at the worksite using direct line-of-sight, cameras, or other visualization devices. Surgical devices, such as the various embodiments of the temporarily positionable devices disclosed herein, may be introduced through the working channel of the endoscope to perform key surgical activities (KSA). Natural orifice endoscopic translumenal procedures developed by Ethicon Endosurgery, Inc. are known in the art as Natural Orifice Translumenal Endoscopic Surgery (NOTES™).

Various embodiments of temporarily positionable devices disclosed herein may be employed in endoscopic, laparoscopic, open surgical procedures, or any combinations thereof. Endoscopy is a minimally invasive surgical procedure vehicle for minimally invasive surgery and refers to looking inside the human body for medical reasons. Endoscopy may be performed using an instrument called an endoscope. Endoscopy is a minimally invasive diagnostic medical procedure used to evaluate the surfaces of organs by inserting a small tube into the body, often, but not necessarily, through a natural body opening or through a relatively small incision or keyhole. Through the endoscope, an operator may observe surface conditions of the organs including abnormal or diseased tissue such as lesions and other surface conditions. The endoscope may have a rigid or flexible tube and, in addition to providing an image for visual inspection and photography, the endoscope may be adapted and configured for taking biopsies, retrieving foreign objects, and introducing medical instruments to a tissue treatment region termed herein as a target site.

Laparoscopic and thoracoscopic surgery belong to the broader field of endoscopy. Laparoscopy also is a minimally invasive surgical technique in which operations in the abdomen are performed through small incisions (usually 0.5-1.5 cm), keyholes, as compared to larger incisions needed in traditional open surgical procedures. Laparoscopic surgery includes operations within the abdominal or pelvic cavities, whereas keyhole surgery performed on the thoracic or chest cavity is called thoracoscopic surgery.

A key element in laparoscopic surgery is the use of a laparoscope, which may be a rigid telescopic rod lens based system, that is usually connected to a video camera (single chip or multi chip) or a distal electronic integrated circuit (chip) based system that places the video camera optics and electronics at the tip of the laparoscope. Also attached to the proximal end of the laparoscope may be a fiber optic cable system connected to a “cold” light source (halogen or xenon) to illuminate the operative field. Alternatively, illumination may be achieved using a solid-state element, such as a light emitting diode (LED) placed at the distal end of the laparoscope. The laparoscope may be inserted through a 5 mm or 10 mm trocar or keyhole to view the operative field. The abdomen is usually insufflated with carbon dioxide gas elevating the abdominal wall above the internal organs like a dome to create a working and viewing space. Carbon dioxide gas is used because it is common to the human body and can be removed by the respiratory system if it is absorbed through tissue.

Various embodiments of minimally invasive temporarily positionable devices described herein may comprise temporarily positionable devices inserted in a patient to provide visualization of the target site. These devices may be introduced into the patient using minimally invasive procedures through natural orifices (e.g., NOTES™ procedures) or via a device inserted through a trocar, for example, and may be adapted to provide images of the worksite or anatomic location such as the lungs, liver, stomach, gall bladder, urinary tract, reproductive tract, and intestinal tissue, for example. Once positioned at the worksite, the temporarily positionable visualization devices provide images that enable the clinician to more accurately diagnose and provide more effective treatment of the diseased tissue. Some portions of the temporarily positionable visualization device may be inserted into the tissue treatment region percutaneously. Other portions of the temporarily positionable visualization device may be introduced into the tissue treatment region endoscopically (e.g., laparoscopically and/or thoracoscopically), through small keyhole incisions via a trocar, or through a natural orifice. Embodiments of the temporarily positionable visualization devices may provide images of the desired tissue during in-vivo treatment procedures used to ablate or destroy live cancerous tissue, tumors, masses, lesions, and other abnormal tissue growths present at the tissue treatment site. Other embodiments of the temporarily positionable visualization devices may be configured to transmit electrical signals to a receiver and then convert the signals into a viewable image. The signals may be transmitted outside the patient either wirelessly or through electrical conductors placed percutaneously or through the same access path as the translumenal endoscopic access device. Other embodiments of the temporarily positionable visualization devices may be powered by on-board power sources, such as a battery, percutaneous electrical conductors, wireless power conductors, or electrical conductors introduced along the same path as the translumenal endoscopic access devices. The embodiments, however, are limited in the context of temporarily positionable visualization and illumination devices.

For example, in various other embodiments, a variety of temporarily positionable end-effector devices may be coupled to a suitable applier and introduced through the flexible working channel of an endoscope introduced inside a patient through a natural opening. Examples of such temporarily positionable end-effectors include, but are not limited to retraction clips, tissue clamps, endoscope stabilizers, electrical power distribution devices, space creators such as devices configured to create space between internal body lumen, organs, and/or dissected sections of tissue, pace makers, vascular access ports, injection ports (such as used with gastric bands), and gastric pacing devices, among other devices.

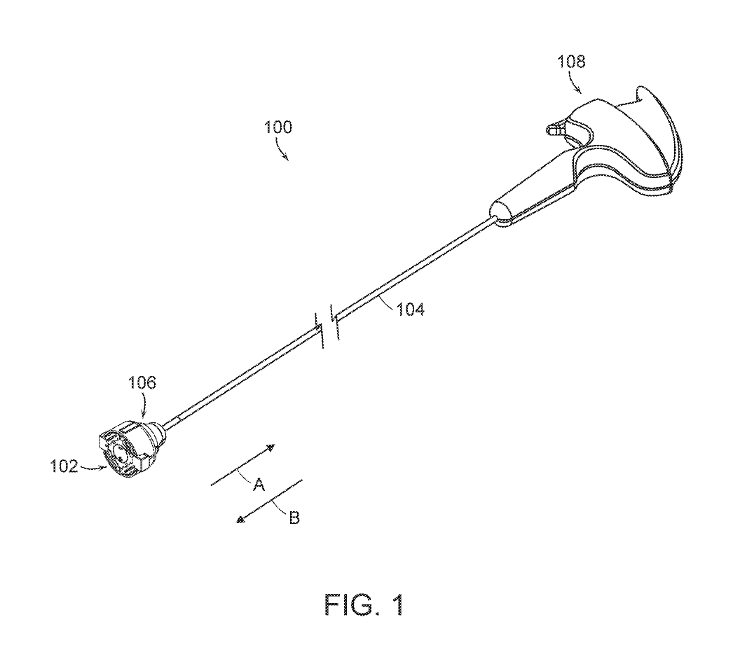

FIGS. 1-5 illustrate one embodiment of a temporarily positionable medical device. The temporarily positionable medical device comprises rearward image acquisition capabilities. In one embodiment, the temporarily positionable medical device comprises an image acquisition system with visualization elements. The temporarily positionable device may be delivered to the worksite using minimally invasive surgical procedures previously described. An attachment mechanism quickly and easily removably secures the device to internal body tissue. The reversible attachment mechanism enables quick and easy attachment, detachment, positioning, repositioning, and/or removal of the temporarily positionable device. The attachment mechanism may be actuated using standard commercially available applier instruments or may be actuated with custom applier instruments. One embodiment of an applier that works in conjunction with the temporarily positionable device is described hereinbelow. The applier may be employed to locate the device at worksite and quickly and easily actuate the attachment mechanism to secure the device to the internal body tissue of the patient.

FIG. 1 is a perspective view of one embodiment of a system for applying a temporarily positionable medical device inside a patient. In one embodiment, the temporarily positionable medical device comprises a visualization system to provide visualization of the patient's anatomy in the direction indicated by arrow “A” once the device is deployed. The rearward viewing mode in direction “A” is used to acquire images while the temporarily positionable medical device is attached to the patient's anatomy. In one embodiment, the temporarily positionable device may be deployed using minimally invasive surgical procedures (e.g., endoscopic, laparoscopic, thoracoscopic, or any combination thereof). In the embodiment illustrated in FIG. 1, a camera applier system 100 comprises a temporarily positionable camera 102, a shaft 104, an applier 106, and a deployment handle 108. In one embodiment, the shaft 104 may be a flexible or articulating tube. The camera 102 may be preloaded into the applier 106. The camera 102 is shown in a preloaded position within the applier 106. The term “camera” may refer to any image visualization device comprising image sensors suitable for capturing light and converting images to electrical signals that can be stored in electronic storage media or transmitted to external devices for displaying the images in real-time. The images may include still photographs or a sequence of images forming a moving picture (e.g., movies or videos). The electrical signals may be transmitted wirelessly or on a wire. Prior to intubating the camera 102 into an endoscopic trocar, the endoscopist (e.g., clinician, physician, or surgeon) inserts the camera 102 into the applier 106 and attaches the preloaded camera 102/applier 106 assembly to the distal end of the shaft 104. The camera 102/applier 106 assembly is then introduced through a flexible endoscopic trocar and is deployed at the desired anatomical location (e.g., worksite or deployment site) inside the patient using an integral attachment mechanism. The camera 102 may be deployed in a desired tissue plane using the integral attachment mechanism. The embodiments, however, are not limited in this context as other techniques may be employed to deliver the camera 102 to the target worksite.

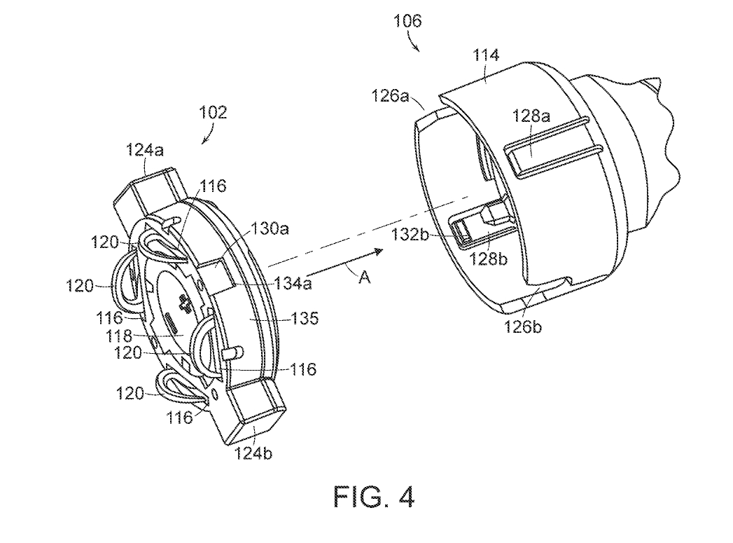

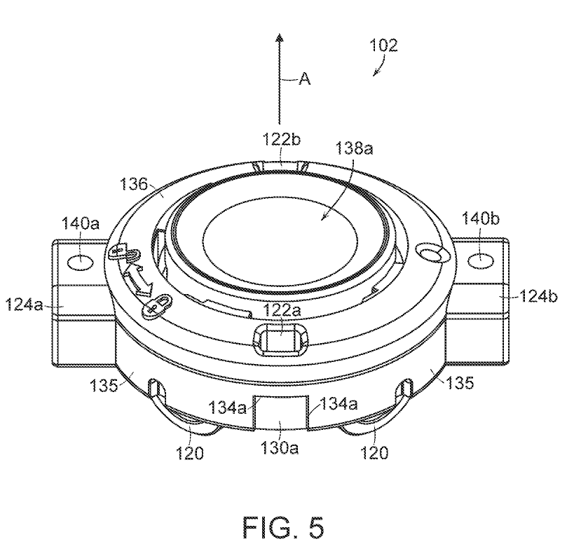

In one embodiment, the applier 106 is suitably configured to receive and contain the camera 102 therein and to couple to the deployment handle 108 via the shaft 104. The shaft 104 is flexible and is suitable for deploying the applier 106 and the camera 102 via the inner working channel of a flexible endoscope, for example. The deployment handle 108 is coupled to the camera 102 via the applier 106 through the shaft 104. In flexible endoscopic translumenal procedures, the flexible/articulating shaft 104 enables the applier 106 to traverse the tortuous paths of the natural openings of the patient through the working channel of a flexible endoscope. For example, the shaft 104 can me made suitably flexible or may comprise articulated elements to make it suitable to traverse the gastrointestinal (GI) tract. In one embodiment, the camera 102 may be positioned within the applier 106 so as to be forward facing in the direction indicated by arrow “B” such that the camera 102 provides visualization feedback while the shaft 104 traverses the GI tract during insertion of the applier 106 and the camera 102 into the patient. Once ready for actuation, the camera 102 may be positioned for deployment. In one embodiment, the camera 102 may comprise multiple active viewing elements or lenses such that the viewing direction “A” or “B” may be selectable. For example one viewing element may be employed for forward viewing in direction “B” during deployment and another viewing element may be employed for backward viewing in direction “A” once deployed. The camera 102 may comprise an attachment mechanism suitable for attaching the camera 102 to the desired tissue at a desired location inside the patient. The attachment mechanism may comprise one or more fasteners 120 (FIG. 4). In the illustrated embodiment, the fasteners 120 are formed as needle-like hooks suitable for penetrating tissue and attaching the camera 102 thereto. The attachment mechanism may be actuated by engaging slots or openings 122a, 122b (FIG. 5) with commercially available instruments or the applier 106. The applier 106 may be configured to deploy, position, reposition, or remove the camera 102. As described with more particularity below, the deployment handle 108 may comprise deployment and reversing triggers to deploy and remove the attachment mechanism once the camera 102 is attached at the desired position. The embodiments, however, are not limited in this context.

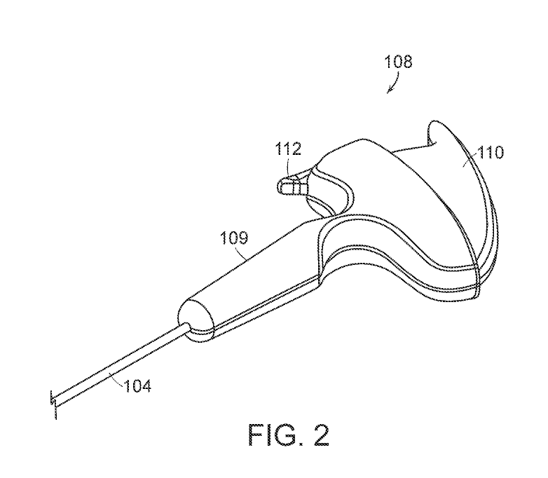

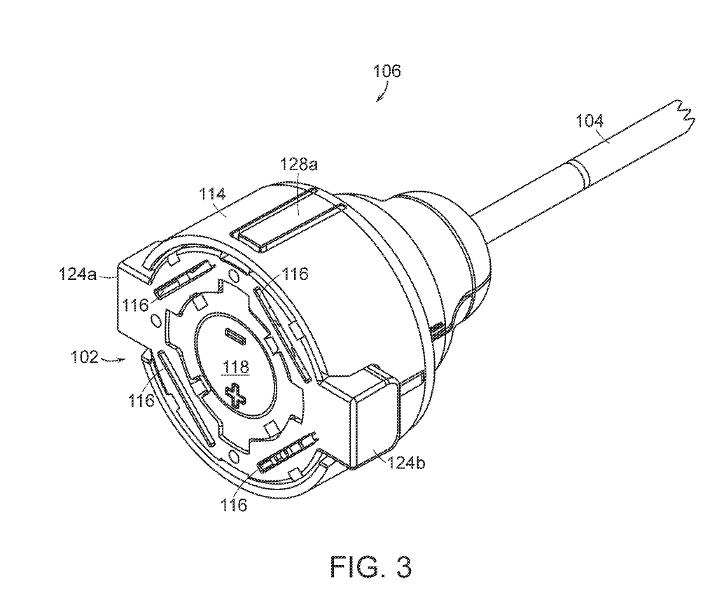

FIG. 2 is a perspective view of one embodiment of a deployment handle for use with the applier and attachment mechanism illustrated in FIG. 1. In the illustrated embodiment, a deployment handle 108 comprises a body 109, a trigger 110 for deployment and reversal, and a lockout button 112. The trigger 110 is actuated to attach the camera 102 (FIG. 1) to the target tissue site. The lockout button 112 prevents unintentional deployment of the attachment mechanism (e.g., the fasteners 120 such as the needle-like hooks illustrated in FIG. 4). The camera 102 may be attached to the tissue by engaging the lockout button 112, e.g. depressing the lockout button 112, and actuating the trigger 110, e.g., by squeezing the trigger 110. Other methods for engaging the lockout button 112 and actuating the trigger 110 are within the scope of this disclosure. The trigger 110 may be configured to lock into place once it is fully engaged or depressed. If the camera 102 is not positioned in a desired location, the clinician may reverse the trigger 110 by depressing the lockout button 112 to re-engage the camera 102 into the camera shroud 114 (FIG. 3). This causes the fasteners 120 to reverse out of the tissue and back into one or more recesses 116 (FIG. 3) formed in the camera 102.

FIG. 3 is a perspective view of one embodiment of a temporarily positionable medical device and applier therefor in a pre-fired position. In the illustrated embodiment, the camera 102 is shown pre-loaded into the shroud 114 and attached to one embodiment of the applier 106. FIG. 4 is an exploded view of one embodiment of the temporarily positionable medical device and applier therefor shown in FIG. 3 in a post-fired released position after being fired from the applier 106. With reference to FIGS. 3 and 4, in one embodiment, the camera shroud 114 portion of the applier 106 comprises cantilever arms 128a, 128b to engage corresponding recesses 130a, 130b (130b not shown) formed with inwardly extending flanges 132a, 132b (132a not shown) to retain the camera 102 in place within the shroud 114. In the pre-fired state shown in FIG. 3, the camera 102 is locked into the shroud 114 and is retained by the flanges 132a, 132b, which are shaped complementarily to the corresponding recesses 130a, 130b formed on a body 135 portion of the camera 102. The flanges 132a, 132b are configured to engage respective ledges 134a, 134b (134b not shown) when the camera 102 is in a retained pre-fired position within the applier 106. The camera 102 may be locked inside the shroud 114 prior to deploying the fasteners 120 into the tissue. The undeployed fasteners 120, as shown in FIG. 3, are nested inside the corresponding recesses 116. In one embodiment, the camera 102 comprises a battery 118 to operate various electrical and/or electromechanical elements of the camera 102. For example, the battery 118 supplies electrical energy to power light sources, image sensor arrays, and motors for orienting, panning, and zooming the image sensor arrays or the associated optics or lenses.

As illustrated in FIG. 4, the fasteners 120 are in a fired or deployed state. The fasteners 120 are deployed to attach the camera 102 to the target tissue site (not shown). The recesses 130a, 130b are formed on the body 135 portion of the camera 102. The recesses 130a, 130b (130b not shown) are configured to engage the corresponding flanges 132a, 132b (132a not shown), which are shaped complementarily to the recesses 130a, 130b. The flanges 132a, 132b engage the respective ledges 134a, 134b (134b not shown) to retain the camera 102 in position within the shroud 114 portion of the applier 106. The body 135 portion of the camera 102 also comprises outwardly extending portions 124a, 124b that are received in corresponding openings 126a, 126b when the camera 102 is in a retained position within the shroud 114 portion of the applier 106.

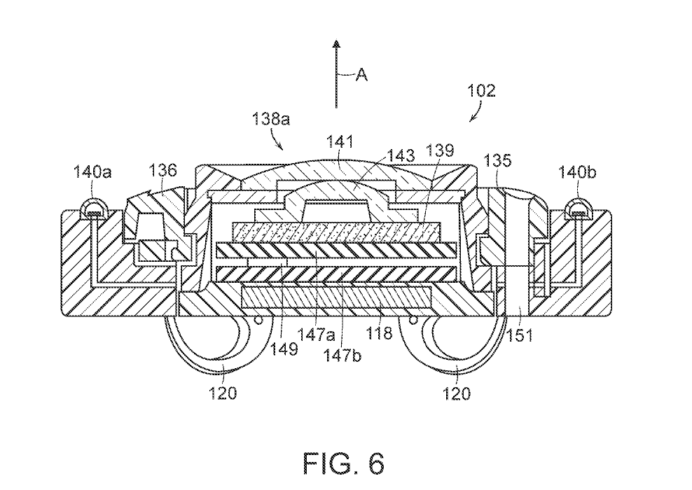

FIG. 5 is a perspective view of one embodiment of a temporarily positionable medical device. In the illustrated embodiment, the camera 102 is shown released or detached from the applier 106. FIG. 6 is a cross-sectional view of the temporarily positionable medical device shown in FIG. 5. With reference to FIGS. 5 and 6, in one embodiment, the camera 102 comprises a body 135 portion, a first lens 138a, a fastener actuator 136, one or more light sources 140a, 140b located in the outwardly extending portions 124a, 124b, openings 122a, 122b to engage an actuator mechanism, one or more fasteners 120, a first image sensor 139, and a battery or plurality of batteries 118. The first lens 138a may be a single optical lens or a system of optical lenses optically coupled to the image sensor 139 contained within the body 135 portion of the camera 102. In one embodiment, the first lens 138a may comprise a lens cap 141 and a first optical lens 143. The lens cap 141 seals the lens 143 and electronic circuitry contained in the body 135 portion of the camera 102 from bodily fluids.

The camera 102 may be employed during natural orifice translumenal endoscopic procedures to provide images of the surgical site that are similar in quality and orientation to those obtainable in open or laparoscopic procedures. For example, in laparoscopic procedures, a laparoscope may be rotated about its optical axis, translated forward and rearward, and may be rotated about a pivot point defined by a trocar or tissue keyhole site to control its orientation and obtain a quality image at a desired viewing angle. During laparoscopic procedures, a clinician can manipulate the laparoscope to provide an optimal image of the surgical site. In addition, the laparoscope can be used to pan and/or zoom the images while the clinician manipulates the laparoscope independently of manipulating tissue or organs proximate to the surgical site.

In one embodiment, the first optical lens 143 may be optically coupled to one or more image sensors 139 to convert an optical image to an electric signal, similar to that employed in digital cameras and other electronic imaging devices. In one embodiment, the image sensor 139 comprises one or more arrays of charge coupled devices (CCD) or complementary metal oxide semiconductor (CMOS) devices such as active-pixel sensors. The image sensor 139 captures light and converts it into electrical signals. A large area image sensor 139 may be used to provide image quality equivalent to that obtainable with standard laparoscopes. In one embodiment, the image sensor 139 may comprise a sensor array with an image input area of approximately 10 mm diameter. Motors may be employed for orienting, panning, and zooming the image sensor 139 and providing an optimal viewing angle of the target anatomy in a desired orientation.

The first image sensor 139 is connected to a first circuit board 147a. The first circuit board 147a also comprises any necessary electronic components or elements for processing, storing, and/or transmitting the images received by the first image sensor 139. The images may be processed by any suitable digital or analog signal processing circuits and/or techniques. Furthermore, the images may be stored in electronic storage media such as, for example, memory devices. The images may be transmitted over a wire or wirelessly to external devices for displaying or further processing the images in real-time. A second circuit board 147b may be employed to receive and attach the battery 118. The first and second circuit boards 147a, 147b are coupled by a connector 149. It will be appreciated by those skilled in the art that a single circuit board or additional circuit boards may be employed without limiting the scope of the illustrated embodiment. The circuit boards 147a, 147b may be formed on a variety of substrates such as printed circuit boards or ceramic substrates and may be connected by one or more connectors 149. A port 151 is provided to receive electrical conductors for carrying image signals or for carrying electric power to the camera 102. The electrical conductors may be removably connected to one or more connectors located on either the first or second circuit board 147a, 147b.

One or more light sources 140a, 140b may be located on the outwardly extending portions 124a, 124b of the body 135 portion to illuminate the site to be imaged. The light sources 140a, 140b may comprise LED based light sources. In one embodiment, the light sources 140a, 140b may comprise a single LED or a combination of LEDs to produce light of a desired spectrum. In other embodiments, fiber optic light sources may be introduced through the working channel of a flexible endoscope. In other embodiments, the light sources 140a, 140b may be coupled to motors for panning and zooming the light sources 140a, 140b in conjunction with the image sensor 139 and provide optimal illumination of the target site.

It will be appreciated by those skilled in the art that the first lens 138a and/or the light sources 140a, 140b may be located on either the front or rear portions of the camera 102. In the embodiment illustrated in FIGS. 1-6, for example, the first lens 138a and the light sources 140a, 140b are oriented for viewing and capturing images in an inward viewing mode in direction “A.” For example, when the camera 102 is deployed and attached at the desired location, the first lens 138a and the light sources 140a, 140b are oriented for viewing the anatomy of the surgical site at a suitable viewing angle and to provide visual feedback during a surgical procedure.

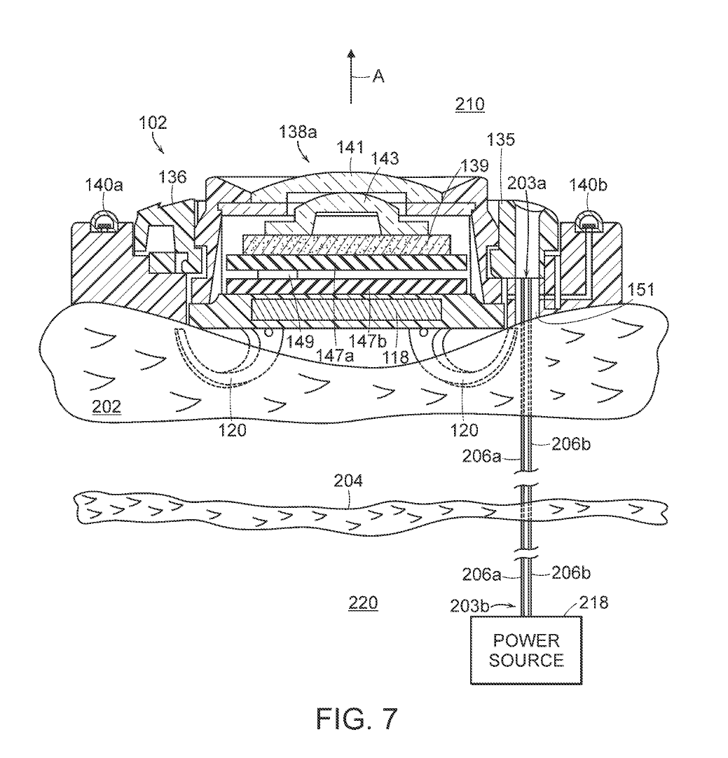

FIG. 7 is a cross-sectional view of the temporarily positionable remote medical device shown in FIG. 5 attached to the abdominal wall with one or more fasteners. In the embodiment illustrated in FIG. 7, a cross-sectional view of one embodiment of the camera 102 is located within the abdominal cavity 210 of a patient and is attached to the abdominal wall 202 with the one or more fasteners 120. The camera 102 is electrically coupled to a medical grade power source 218 via insulated percutaneous electrical conductors 206a, 206b. The percutaneous electrical conductors 206a, 206b supply electrical energy to the camera 102 and/or transmit image signals between the camera 102 and an external monitor. Although shown as single individual conductors, those skilled in the art will appreciate that each of the percutaneous electrical conductors 206a, 206b may comprise multiple insulated conductors within an insulative sleeve. A first end 203a of the percutaneous electrical conductors 206a, 206b is connected to the camera 102 and a second end 203b is connected to the power source 218. The percutaneous electrical conductors 206a, 206b comprise an outer electrically insulative sleeve having a total outside diameter suitable for penetrating the skin 204 and the abdominal wall 202 without requiring special closure procedures. In one embodiment, the total outside diameter of the insulative sleeve may be less than approximately 17 gauge. As illustrated in FIG. 7, the percutaneous electrical conductors 206a, 206b are inserted through the skin 204 and the abdominal wall 202, and are received into the port 151 to couple to the camera 102. In one embodiment, the percutaneous electrical conductors 206a, 206b may be rigidly connected to the camera 102 to enable a clinician to position the camera 102 by manipulating the percutaneous electrical conductors 206a, 206b from outside the patient. In one embodiment, the percutaneous electrical conductors 206a, 206b may be removably connected to the camera 102. Introducing the percutaneous electrical conductors 206a, 206b through the skin 204 frees up space in the working channel of the endoscope for additional surgical instruments.

In one embodiment, the power source 218 may be a low voltage direct current (DC) power supply. The power source 218 may be located outside the abdominal wall 202 or may be located in an area 220 outside of the patient. The first and second percutaneous electrical conductors 206a, 206b can be used to supply power to the camera 102 and/or to other surgical devices and accessories. Alternately, the camera 102 may be coupled to an external monitor via the first and second percutaneous electrical conductors 206a, 206b. In one embodiment, the camera 102 may be powered by the external power source 218, the battery 118, or a combination thereof. The external power source 218 is particularly useful when the camera 102 is equipped with the one or more light sources 140a, 140b, the image sensor 139 array, and one or more motors for positioning the image sensor 139 array, which in combination may require more power than can be delivered by the battery 118 alone. The power source 218 may be configured to supply power to other deployable and undeployable surgical devices and accessories.

In some implementations, insulated electrical conductors may be introduced through the working channel of an endoscope. In one embodiment, electrical conductors may be removably attached to the camera 102 during the delivery and deployment phases, as may be typical in natural orifice translumenal endoscopic procedures. The removably attachable conductors may be delivered to the camera 102 either through the working channel of a flexible endoscope or along side of the scope. Once the camera 102 is deployed, the removably attachable conductors may be disconnected from the camera 102 and retrieved through the working channel or along side of the endoscope. During the delivery and deployment phases, the camera 102 initially may be removably coupled to the power source 218 with the removably attachable conductors. Once the camera 102 is deployed, the removably attachable conductors may be disconnected from the camera 102 and the percutaneous conductors 206a, 206b may be connected to the camera 102 to establish power from the power source 218.

In one embodiment, the camera 102 may comprise a wireless component for wirelessly transmitting images outside the patient. The wireless component may be a radio frequency (RF) device suitable for transmitting images remotely from the patient to an external monitor. The wireless component may be powered either by the battery 118 or by the power source 218 through the percutaneous electrical conductors 206a, 206b. In one embodiment, the wireless component may comprise a wireless transceiver (e.g., RF transmitter and receiver) module. Images received by the image sensor 139 may be wirelessly transmitted/received between the wireless RF device using any well known RF telemetry techniques so as to eliminate the need for hard wired electrical connections.

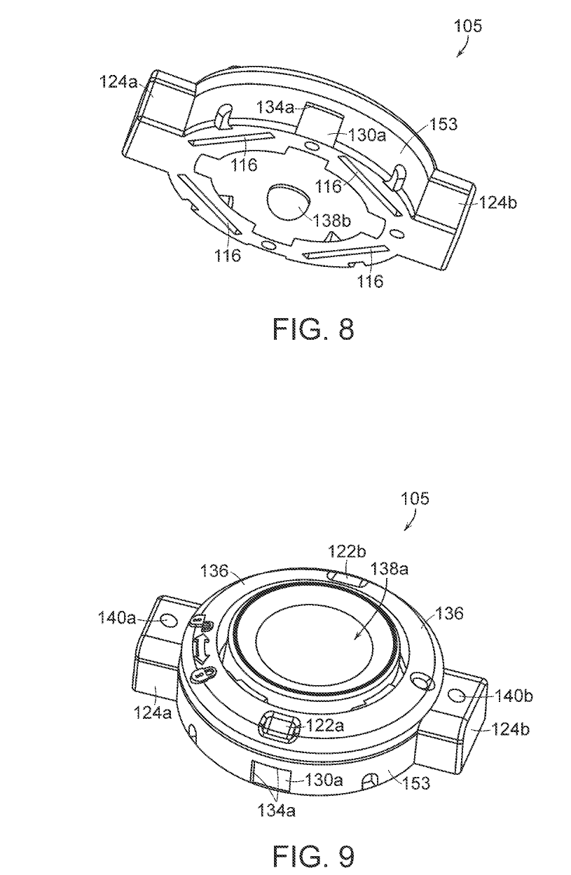

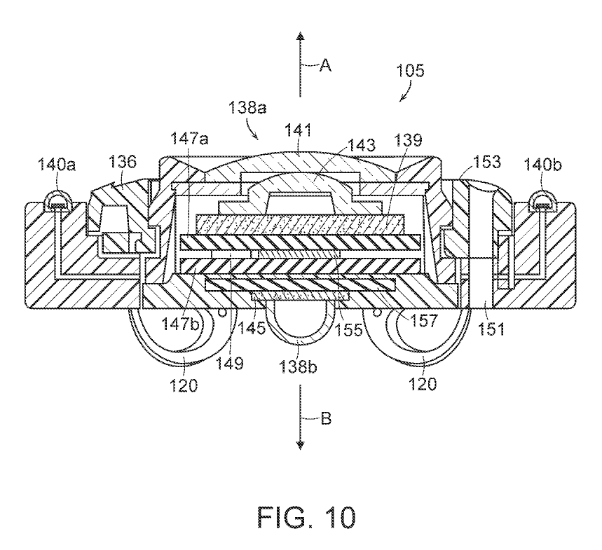

FIG. 8 is a perspective view of one embodiment of a forward and rearward viewing temporarily positionable medical device released from an applier showing a forward image acquisition portion. FIG. 9 is a perspective view of one embodiment of the forward and rearward viewing temporarily positionable medical device shown in FIG. 8 released from an applier showing a rearward image acquisition portion. FIG. 10 is a cross-sectional view of one embodiment of the forward and rearward viewing temporarily positionable medical device shown in FIGS. 8 and 9. With reference to FIGS. 8-10, in one embodiment, a camera 105 comprises a body 153, the first lens 138a, a second lens 138b, the fastener actuator 136, the one or more light sources 140a, 140b located in the outwardly extending portions 124a, 124b, openings 122a, 122b to engage an actuator mechanism, the one or more fasteners 120, the first image sensor 139, a second image sensor 145, and a battery 155. The first lens 138a may be a single optical lens or a system of optical lenses optically coupled to the first image sensor 139 contained within the body 153 portion of the camera 105, as previously discussed with respect to the camera 102. The second lens 138b may be a single optical lens or system of optical lenses optically coupled to a second image sensor 145 contained within the body 153 portion of the camera 105. In one embodiment, the second optical lens 138b may be optically coupled to the second image sensor 145. The second image sensor 145 captures light and converts it into electrical signals similar to that employed in digital cameras and other electronic imaging devices. In one embodiment, the second image sensor 145 comprises one or more arrays of CCDs or CMOS devices such as active-pixel sensors.

In the illustrated embodiment, the second lens 138b is located on a side opposite to that of the first lens 138a. In typical natural orifice translumenal endoscopic procedures, the second lens 138b is used in a forwarding viewing mode in direction “B” during the delivery and deployment phases of the camera 105 to guide the applier and the camera 105 to the worksite. The second image sensor 145 is suitable for capturing light and converting images to electrical signals that can be stored in electronic storage media or transmitted to external devices for displaying the images in real-time. The electrical signals can be transmitted on a wire or wirelessly.

The body 153 portion of the camera 105 comprises recesses 116 to contain the nested undeployed fasteners 120 similar to those previously discussed with respect to the camera 102. The recesses 130a, 130b (130b not shown) are configured to engage corresponding flanges 132a, 132b formed on the camera shroud 114 portion of the applier 106 as previously discussed with respect to FIGS. 3 and 4. The flanges 132a, 132b engage the respective ledges 134a, 134b (134b not shown) to retain the camera 105 in position within the shroud 114 portion of the applier 106. The body 153 portion of the camera 105 also comprises outwardly extending portions 124a, 124b that are received in corresponding openings 126a, 126b of the shroud 114 (FIGS. 3 and 4) when the camera 105 is in a retained position within the shroud 114 portion of the applier 106.

As previously discussed, the first image sensor 139 is connected to the first circuit board 147a, which also comprises any necessary electronic components for processing, storing, and/or transmitting the images received by the first image sensor 139. The battery 155 is connected to the second circuit board 147b. The first and second circuit boards 147a, 147b are coupled by a connector 149. It will be appreciated by those skilled in the art that a single circuit board or additional circuit boards may be employed without limiting the scope of the illustrated embodiment. The circuit boards 147a, 147b may be formed on a variety of substrates such as printed circuit boards or ceramic substrates and may be connected by one or more connectors 149. The port 151 is provided to receive electrical conductors to carry image signals or to carry electrical power to the camera 105. The electrical conductors may be removably connected to one or more connectors located on either the first or second circuit board 147a, 147b.

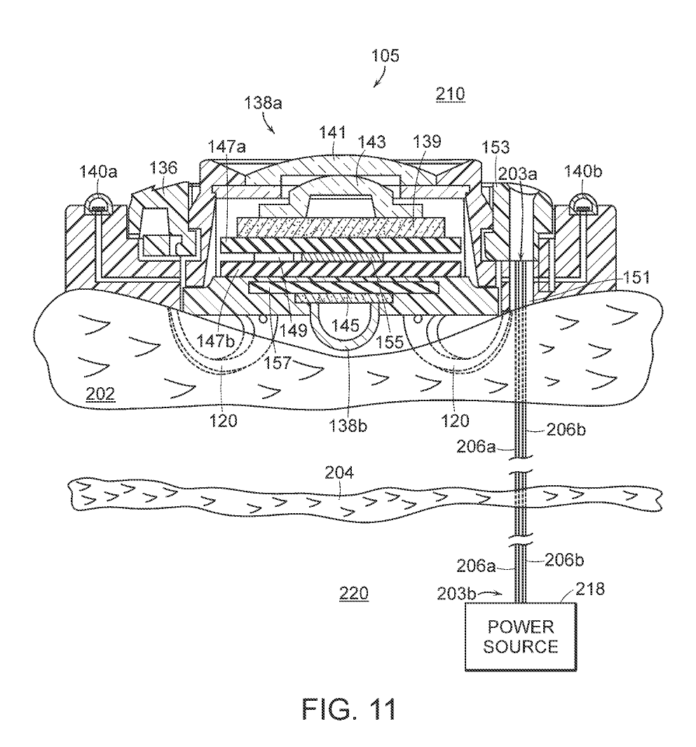

FIG. 11 is a cross-sectional view of one embodiment of the temporarily positionable medical device shown in FIG. 10 attached to the abdominal wall with one or more fasteners. In the embodiment illustrated in FIG. 11, the camera 105 is attached to the abdominal wall 202 within the abdominal cavity 210 of a patient. The camera 105 is electrically coupled to the medical grade power source 218 via insulated percutaneous electrical conductors 206a, 206b, as previously discussed with reference to FIG. 7.

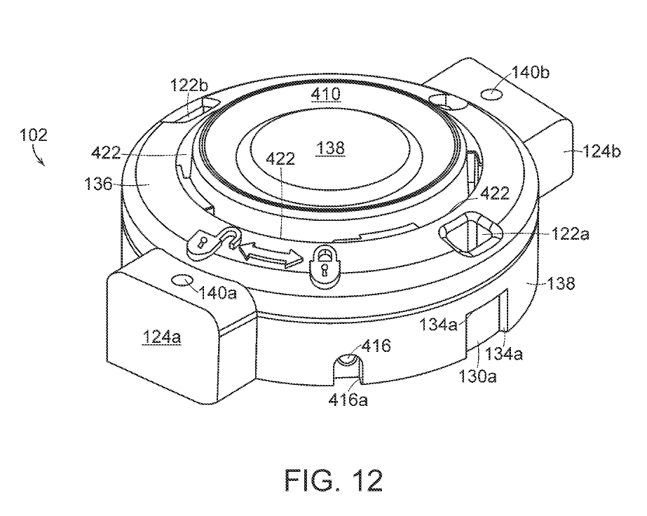

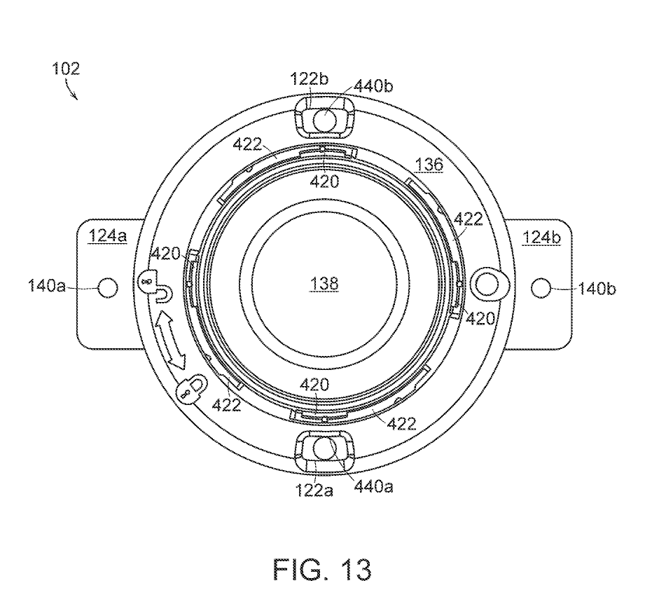

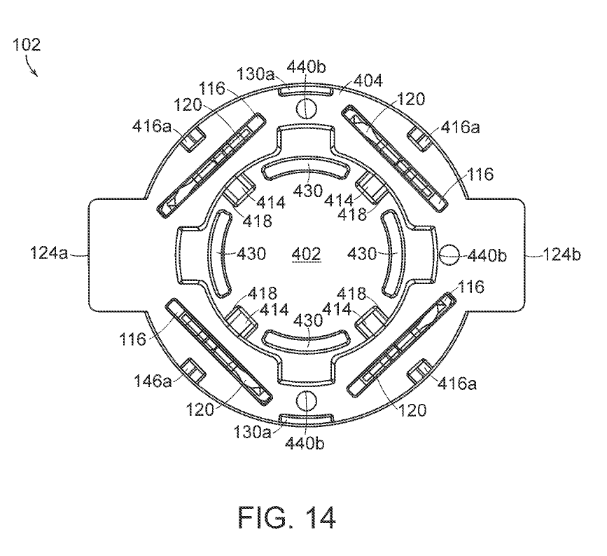

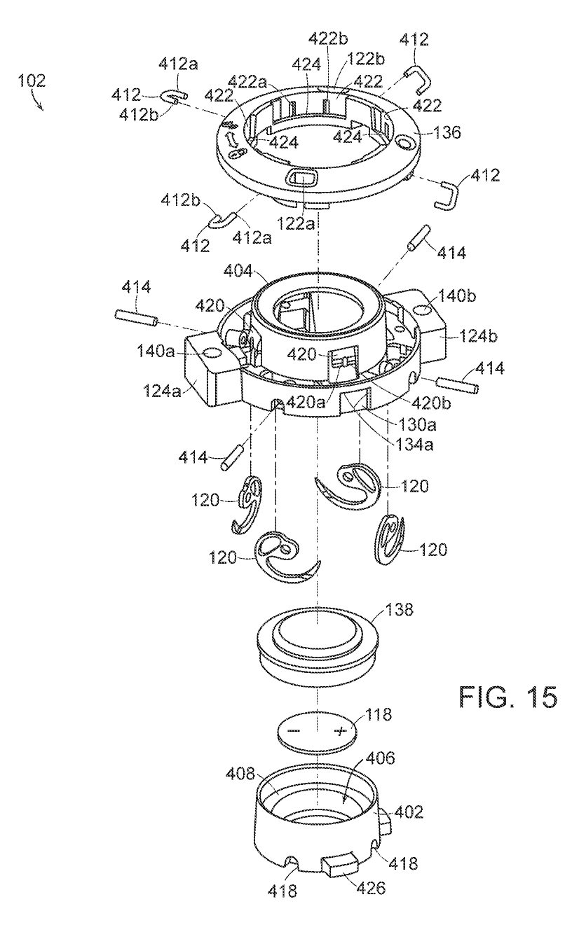

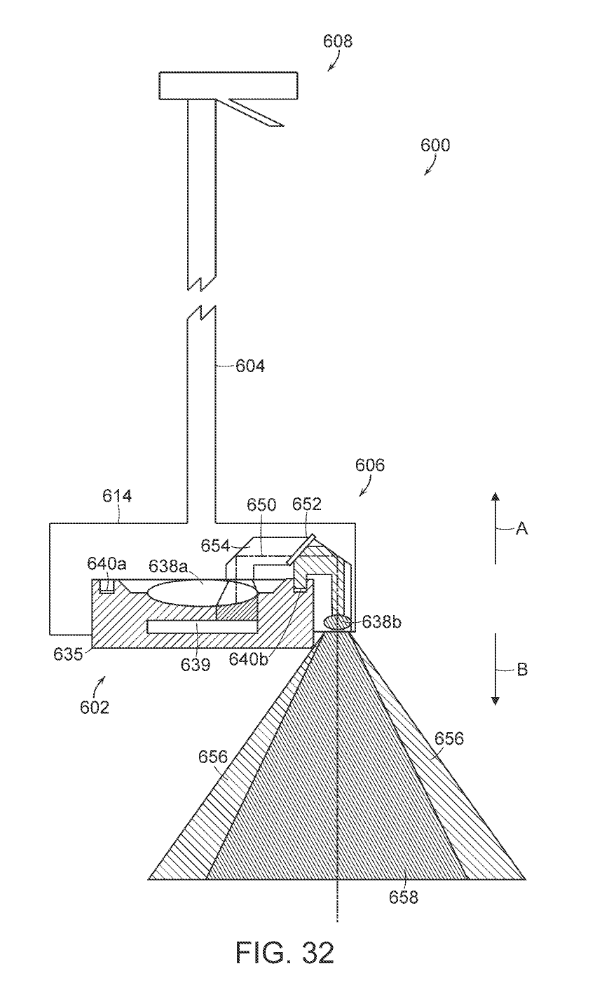

FIGS. 12-15 illustrate one embodiment of a temporarily positionable medical device comprising a rearward image acquisition portion and an integral attachment mechanism. In particular, FIG. 12 is a perspective view of one embodiment of a temporarily positionable medical device showing a rearward image acquisition portion. FIG. 13 is a top view of the embodiment of the temporarily positionable medical device shown in FIG. 12. FIG. 14 is a bottom view of one embodiment of the temporarily positionable medical device shown in FIG. 12. And FIG. 15 is an exploded perspective view of one embodiment of the temporarily positionable medical device shown in FIG. 12. In the embodiment illustrated in FIGS. 12-15, the camera 102 comprises a rearward image acquisition portion and an integral attachment mechanism. The attachment mechanism may be used with any temporarily positionable or implantable medical device for which it is suited including, by way of example, forward and rearward viewing cameras similar to the camera 105 previously discussed, retraction clips, clamps, scope stabilizers, power distributors, space creators, pace makers, vascular access ports, injection ports (such as used with gastric bands), and gastric pacing devices. Several of these embodiments are described with particularity in FIGS. 32-37 below.

With reference to FIGS. 12-15, in one embodiment, the camera 102 includes the lens 138, a lens retainer 402, and a camera body 404. The camera 102, with integral attachment mechanism, also includes the fasteners 120, the fastener actuator 136, and a plurality of link members 412.

The lens 138 may be made of any biocompatible material having suitable optical properties such as Polycarbonate or silica glass. The lens 138 is disposed partially within an internal cavity 406 of the lens retainer 402 adjacent to an annular flat 408. The lens retainer 402, the camera body 404, and the fastener actuator 136 may be made of any suitable biocompatible material having sufficient stiffness and strength such as polyetheretherketon (known as PEEK). The fasteners 120 and the link members 412 may be made of any suitable biocompatible material such as stainless steel.

The camera body 404 includes an annular rim 548 that engages the upper surface of the lens 138 about an annular portion. The camera body 404 is retained to the lens retainer 402 by a plurality of pins 414 that are disposed through respective holes 416 formed in recesses 416a in the camera body 404 and extend inwardly into the respective recesses 418 formed about the bottom periphery of the lens retainer 402. The pins 414 may be made of any suitable biocompatible material, such as stainless steel.

The fastener actuator 136 is secured to the camera body 404. Although in the illustrated embodiment the fastener actuator 136 is shown as an annular ring rotatably supported by the camera body 404, the fastener actuator 136 may be formed in any suitable configuration and supported in any suitable manner to permit the fastener actuator 136 to move the fasteners 120 between and including deployed and undeployed positions. As shown in FIG. 15, the camera body 404 includes a plurality of downwardly and outwardly extending tabs 420. In the illustrated embodiment, there are four equally spaced tabs 420. The fastener actuator 136 includes an equal number of the corresponding recesses 422, each having an arcuate bottom 424. To assemble the fastener actuator 136 to the camera body 404, the recesses 422 are aligned with the tabs 420, and pushed down, temporarily deflecting the tabs 420 inwardly until the tabs 420 reach the recesses 422 and move outwardly to dispose the lower edges 420a in the recesses 422 such that the fastener actuator 136 is retained thereby. The lengths of the tabs 420 and the depth of the recesses 422 allow some axial end play between the fastener actuator 136 and the camera body 404, as will be described below.

The fastener actuator 136 may rotate generally about the central axis of the camera body 404. In the illustrated embodiment, the fastener actuator 136 may rotate through an angle of about 40 degrees, although any suitable angle may be used. In the illustrated embodiment, when the fastener actuator 136 is rotated in the deploying direction, causing the fasteners 120 to move to the deployed position, rotation of the fastener actuator 136 beyond the fully deployed position is limited by the end 422c contacting tab 420.

A detent system is formed by a pair of spaced apart raised detent ribs 422a, 422b extending inwardly from the wall of each the recess 422 and a corresponding raised rib 420b extending outwardly from the tab 420. The detent system assists in preventing the fastener actuator 136 from rotating and the fasteners 120 from moving out of fully retracted or fully extended fired states under vibration or incidental loads, as described below.

The fastener actuator 136 includes a plurality of spaced apart openings 122a, 122b that may be engaged by any suitable instrument to transmit the necessary torque to the fastener actuator 136 to extend the fasteners 120 to the actuated position. The openings 122a, 122b are configured to be engaged by commercially available instruments, rectangular in the illustrated embodiment, or by a dedicated applier described below. The camera body 404 includes a plurality of recesses 130a, 130b disposed about its lower periphery. The recesses 130a, 130b are configured to cooperate with the dedicated applier 106 as described below.

Referring to FIGS. 13 and 14, the fastener actuator 136 includes openings 440a formed therethrough that align with corresponding openings 440b formed in the camera body 404 when the fastener actuator 136 is in the undeployed position. The openings 440a and 440b may be used by the clinician to suture the camera 102 if the integral attachment mechanism is not used.

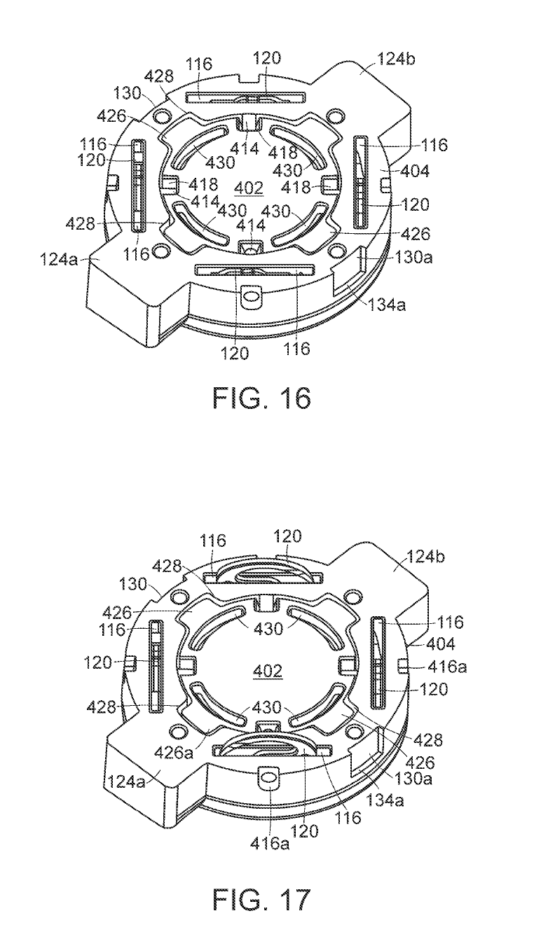

FIGS. 16-17 illustrate one embodiment of the lens retainer 402 including a plurality of locating tabs 426 extending outwardly from adjacent the bottom periphery of the lens retainer 402. Referring to FIGS. 12-17, the locating tabs 426 and 426a are located in respective complementarily shaped recesses 428 formed in the inner surface of the camera body 404, aligning the lens retainer 402 properly with the camera body 404.

FIG. 16 is a bottom perspective view of one embodiment of the temporarily positionable medical device shown in FIG. 12 with fasteners located in a retracted position. In the illustrated embodiment, the temporarily positionable camera 102 is shown with the fasteners 120 located in a retracted position. As illustrated, the fasteners 120 are disposed in respective recesses or slots 116 formed in the camera body 404. FIG. 17 is a bottom perspective view of one embodiment of the temporarily positionable medical device shown in FIG. 12 with fasteners located in an extended, or fired, position, extending from corresponding slots. In the illustrated embodiment, the temporarily positionable camera 102 is shown with the fasteners located in an extended, or fired, position, extending from the corresponding slots 116. Rotation of the fastener actuator 136 moves the fasteners 120 from the retracted position to the extended position.

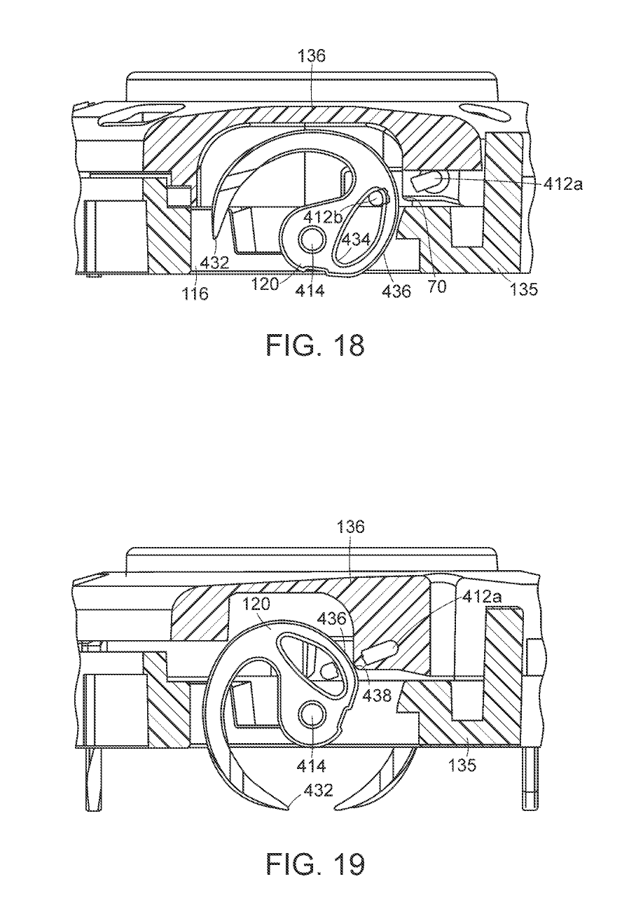

FIGS. 18-21 are a series of figures illustrating the operation of one embodiment of the fastener actuator 136 and one embodiment of the plurality of fasteners 120. It should be understood that the operation of one of the fasteners 120 may be the same as for all the fasteners 120, which may, in one embodiment, be moved from a deployed position to an undeployed position simultaneously.

FIG. 18 is a cross-sectional view of one embodiment of a fastener in a fully retracted state, the undeployed position, disposed completely within a slot such that a sharp tip is not exposed. In the illustrated embodiment, the fastener actuator 136 shows the fastener 120 in a fully retracted state, the undeployed position, disposed completely within the slot 116 such that a sharp tip 432 of the fastener 120 is not exposed. This prevents the sharp tip 432 of the fastener 120 from accidentally sticking the clinician or penetrating any object during the deployment phase. The fastener actuator 136 is illustrated rotated counterclockwise as far as permitted by the recesses 422 and the tabs 420. In this position, a set of raised ribs 420b are disposed clockwise of a second set of detent ribs 422b. The first ends 412a of the link members 412 are rotatably carried by the fastener actuator 136, spaced apart at positions corresponding to the positions of the fasteners 120. The second ends 412b are disposed within openings or slots 434 of the fasteners 120.

To actuate the attachment mechanism, the integral fastener actuator 136 is rotated in a deploying direction, which in the illustrated embodiment is clockwise (any suitable direction configured to actuate the attachment mechanism may be used), and a first raised rib 420b passes a second detent rib 422b, which may produce an audible signal in addition to a tactile signal to the clinician. The second end 412b of the link member 412 is free to move within the slot 434 during actuation, as the force that rotates the fastener 120 into the extended position is transmitted to the fastener 120 through the interaction between a cam surface 436 of the fastener 120 and an actuating cam surface 438 of the fastener actuator 136. As the fastener actuator 136 rotates clockwise, the actuating cam surface 438 engages and pushes against the cam surface 436, rotating the fastener 120 about the pivot pin 414. The majority of the force from the actuating cam surface 438 acts tangentially on the fastener cam surface 436, off center relative to the pivot pin 414, causing the fastener 120 to rotate. During actuation, the end 412b of the link member 412 remains free to move within the slot 434, applying no driving force to rotate the fastener 120.

Referring to FIG. 18, when the fasteners 120 reach the fully undeployed position the tip 432 may be disposed fully in the slot or recess 116. Further undeploying rotation of the fastener actuator 136 is prevented by the link member 412 which is prevented from further movement by the fastener 120.

FIG. 19 is a cross-sectional view of one embodiment of a fastener rotated about half way through its range of rotation, about 90 degrees as a result of a clockwise rotation of an actuator. In the illustrated embodiment, the fastener 120 is rotated about half way through its range of rotation, about 90 degrees as a result of a clockwise rotation of the actuator 136. As the fastener actuator 136 is rotated clockwise, the force between the actuator cam surface 438 and the fastener cam surface 436 causes the fastener actuator 136 to move upward slightly as allowed by the tolerancing of the components. As the fastener actuator 136 is rotated further clockwise from the position shown in FIG. 19 the actuator cam surface 438 continues to engage and push against the fastener cam surface 436, rotating the fastener 120 further counterclockwise.

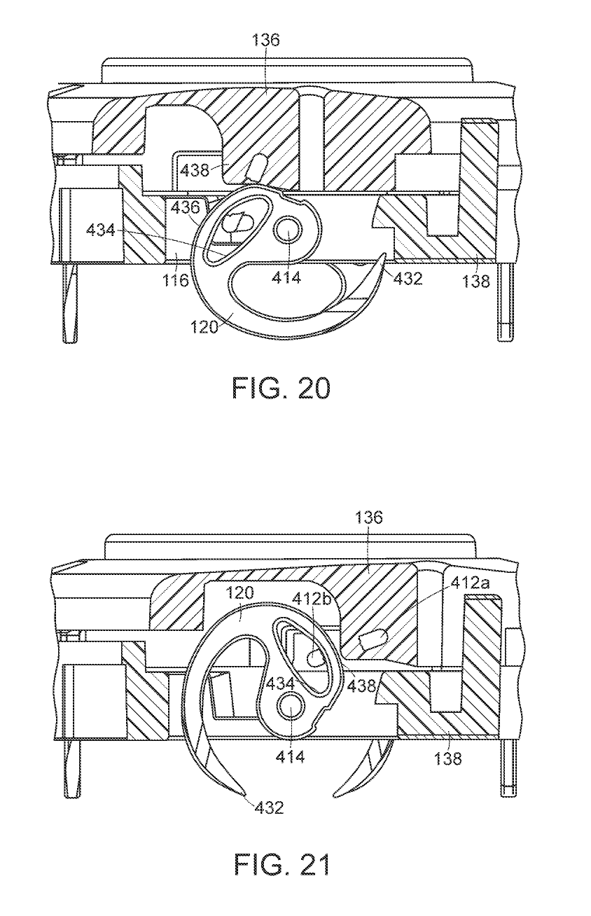

FIG. 20 is a cross-sectional view of one embodiment of a fastener actuator rotated clockwise to its fullest extent, with a raised rib having been urged past the detent rib. In the illustrated embodiment, the fastener actuator 136 is rotated clockwise to its fullest extent, with the raised rib 420b having been urged past the detent rib 422a. In this position, the fastener 120 has rotated to its fullest extent, almost 180 degrees in the illustrated embodiment, with the tip 432 disposed within the recess 116. In this position, the actuator cam surface 438 is over center, and the fastener actuator 136 is resistant to being back driven by an undeploying force imparted to the fastener 120 as the cam surface 436 acts against the actuator cam surface 438 in a direction that tends to push the fastener actuator 136 up instead of rotating the actuator 136. The distal end portion of the fastener 120 is configured essentially as a beam, depicted as having a generally rectangular cross section along its length, tapering to the sharp tip 432. With the fastener 120 extending approximately 180 degrees in the fully extended state, the deployed position, forces which may act on the fasteners 120 tend to act through the pivot axis defined by the pivot pin 414, instead of rotating the fasteners 120. It is noted that although the pivot pin 414 is illustrated as being a separate piece from the fastener 120, the two may be integral or even of unitary construction.

If it is desirable to retract the fasteners 120, such as to remove or reposition the temporarily positionable medical device (e.g., the camera 102 in the illustrated embodiment), the fastener actuator 136 may be rotated in an undeploying direction, counterclockwise in the illustrated embodiment. Starting with the position of the fastener actuator 136 shown in FIG. 20 the fastener actuator 136 may be rotated counterclockwise, with the actuator cam surface 438 sliding against the cam surface 436, without rotating the fastener 120. In the illustrated embodiment, continued counterclockwise rotation of the fastener actuator 136 moves the cam surface 438 out of contact with the cam surface 436, with no substantial rotating force being exerted on the fastener 120 until second end 412b of the link member 412 reaches a location in the slot 434, such as at one end of the slot 434, at which the link member 412 begins pulling against the slot 434 causing the fastener 120 to rotate and begin to retract.

FIG. 21 is a cross-sectional view of one embodiment of the fastener actuator 136 that has been advanced counterclockwise compared to the position shown in FIG. 20, and a fastener rotated approximately halfway through its range. As shown by comparing FIG. 20 to FIG. 21, the fastener actuator 136 is in different positions with the fastener 120 in the same position, in dependence upon whether the attachment mechanism is being actuated or deactuated (retracted). This results from the lost motion that results when the link member 412 is pulling on the slot 434 in comparison to the actuator cam surface 438 pushing directly on the cam surface 436. To retract the fasteners 120 fully, the fastener actuator 136 is rotated until the raised rib 420b snaps past the detent rib 422b.

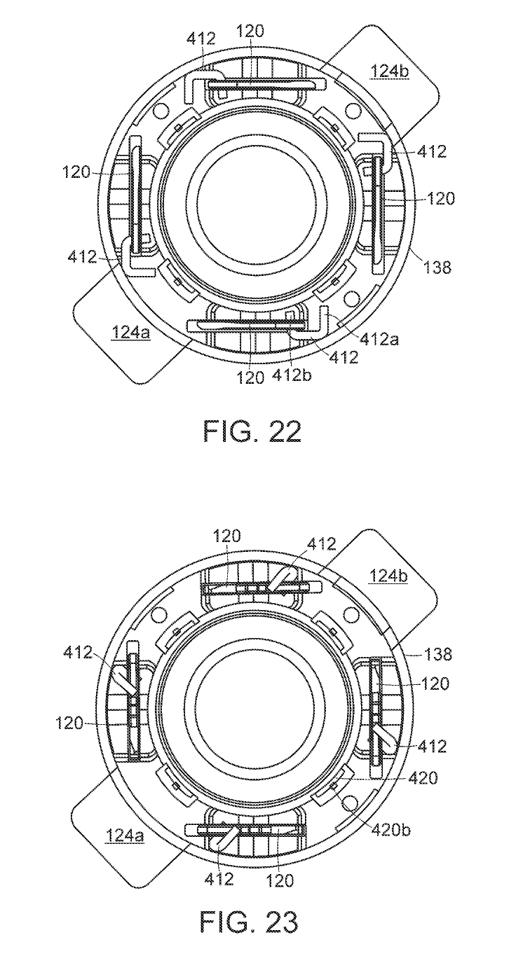

FIG. 22 is a top view of one embodiment of a temporarily positionable medical device with the actuator omitted to illustrate the positions of the links when the fasteners are in the retracted position. In the illustrated embodiment, the temporarily positionable camera 102 is shown with the fastener actuator 136 omitted to illustrate the positions of the link members 412 when the fasteners 120 are in the retracted position. FIG. 23 is a top view of one embodiment of a temporarily positionable medical device with the actuator omitted to illustrate the positions of the links when the fasteners are in the extended/fired position. In the illustrated embodiment, the temporarily positionable camera 102 is shown with the fastener actuator 136 omitted to illustrate the positions of the link members 412 when the fasteners 120 are in the extended/fired position. The link members 412 are shown in their actual positions when the first ends 412a are supported by the fastener actuator 136, in the deployed and in the undeployed states.

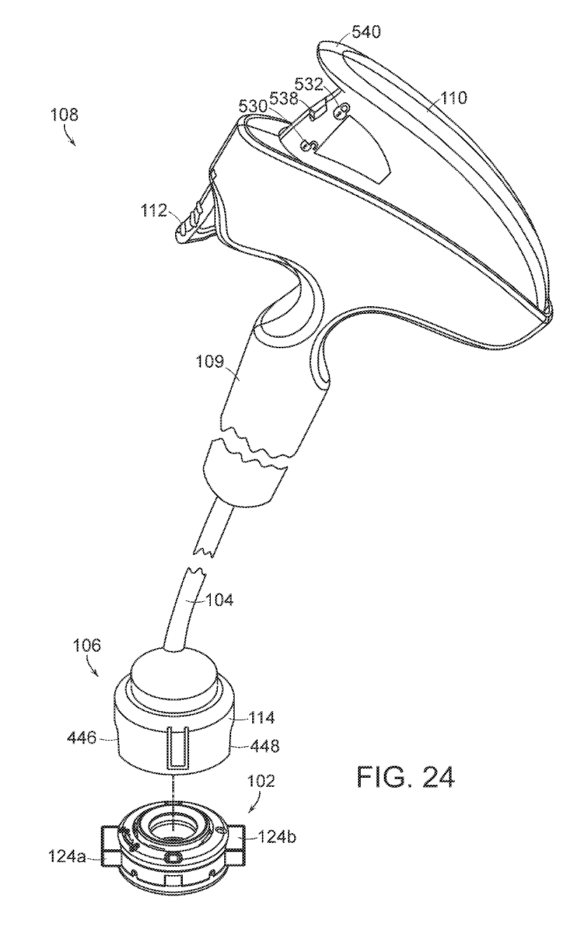

As mentioned previously, the attachment mechanism may be actuated by engaging the openings 122 with commercially available instruments or by a dedicated applier. FIG. 24 illustrates a perspective view of one embodiment of a deployment handle and applier configured to position, actuate, deactuate, remove, or reposition a temporarily positionable medical device through a flexible shaft. In the illustrated embodiment, the deployment handle 108 and the applier 106 are configured to position, actuate, deactuate, remove, or reposition the temporarily positionable camera 102 through the flexible shaft 104. It is noted, however, that the practice of aspects of the deployment handle 108 is not limited to the specific applier illustrated embodiment herein. The deployment handle 108 may be used with any one of the temporarily positionable or implantable medical devices disclosed herein including, but not limited to, forward and/or rearward viewing cameras, retraction clips, clamps, scope stabilizers, power distributors, space creators, pace makers, vascular access ports, injection ports (such as used with gastric bands), and gastric pacing devices, several of which are described with particularity in FIGS. 32-37 below.

As shown in FIG. 24, the deployment handle 108, the applier 106, and the flexible shaft 104 are constructed to position one embodiment of the camera 102 inside a patient using a flexible endoscopic procedure. In the illustrated embodiment, the deployment handle 108 includes the body 109, the camera shroud 114, the trigger 110, and the lockout button 112. As will be described below, the camera 102 may be assembled to the camera shroud 114 with the outwardly extending portions 124a, 124b disposed in the respective alignment slots 446, 448. The camera shroud 114 is angled relative to the body 109, allowing for easier and better visualization of the camera 102 during positioning. In the illustrated embodiment, the angle is 20 degrees and the shaft portion of the body 109 is about 10 cm.

The trigger 110 may include a visual indicator to indicate whether the trigger 110 is fully in the undeployed state, such as an unlocked lock icon 530, and indicia to indicate whether the trigger 110 is in the deployed state, such as a locked lock icon 532. Such visual indication may be include by any suitable manner, such as by molding integral with the trigger 110, applying as a adhesive film or such, or printing directly on the trigger 110. With the indicator 110, the unlocked lock icon may be visible adjacent the upper edge of the body 109, although other configurations of indication may be utilized, such as a window or such formed in the body 109 to reveal the indicia.

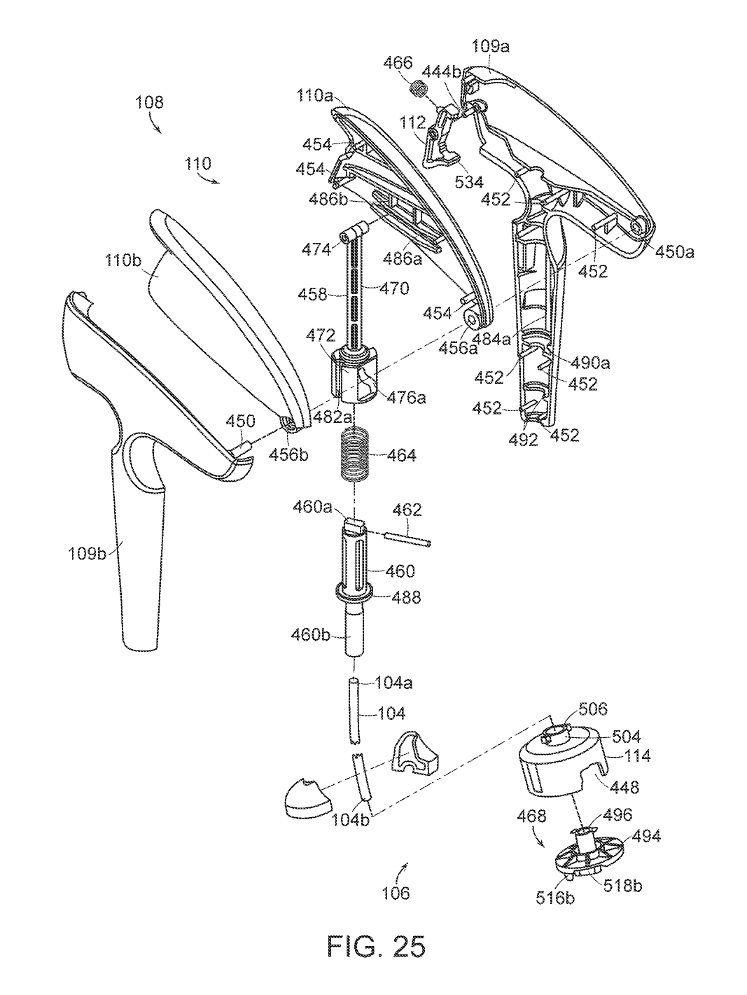

FIG. 25 is an exploded perspective view of one embodiment of the deployment handle, applier, and flexible shaft shown in FIG. 24. In the illustrated embodiment, the body 109 includes first and second halves 109a, 109b assembled to each other to contain the internal components. Except for locating pins 452, the pivot pins 450, and the ship laps, the first and second body halves 109a, 109b are substantially similar to each other. The locating pins 452, illustrated as extending from the first body half 109a, fit into respective complementarily shaped openings (not illustrated) on the second body half 109b. The engagement of a plurality of the locating pins 452 in the openings is sufficient to hold the first and second body halves 109a, 109b together. The pins 452 may alternatively extend from the second body half 109b with the openings carried by the first body half 109a. Any suitable configuration may be used to assemble and secure the first and second body halves 109a, 109b together.

The trigger 110 includes first and second halves 110a, 110b. Locating pins 454, illustrated as extending from the first actuator half 110a, fit into respective complementarily shaped openings (not illustrated) on the second actuator half 110b. The pins 454 may alternatively extend from the second actuator half 110b with the openings carried by the first actuator half 110a. Any suitable configuration may be used to assemble and secure the first and second trigger halves 110a, 110b together. The second body half 109b includes the pivot pin 450 which rotatably supports the trigger 110 at one end, extending through first and second pivot holes 456a, 456b into the opening 450a. The first body half 109a includes a pivot pin 444, which rotatably supports the safety switch 112. The first and second body halves 109a, 109b, the camera shroud 114, the first and second trigger halves 110a, 110b, and the safety switch 112 may be made of any biocompatible material such as polycarbonate. The safety switch 112 is rotated about the pivot pin 444, withdrawing the lockout tab 194 from the lower opening 536, allowing the trigger 110 to be rotated about the pivot pin 414. This action causes the cam track 486 to move the cross member 474 downward, causing the cam collar 472 to rotate the drive shaft 460, thereby rotating the actuator mechanism 468 relative to the camera shroud 114. Rotation of the actuator mechanism 468 actuates the fastener actuator 136 by rotating it. The engagement between the outwardly extending portions 124a, 124b and the respective slots 446, 448, prevent the camera body 404 from rotating, allowing relative motion between the fastener actuator 136 and the camera body 404.

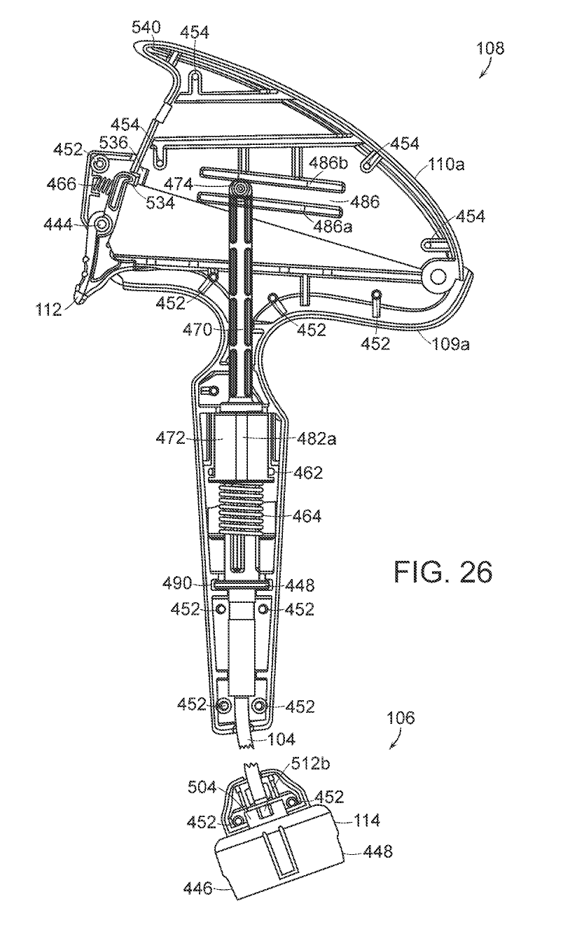

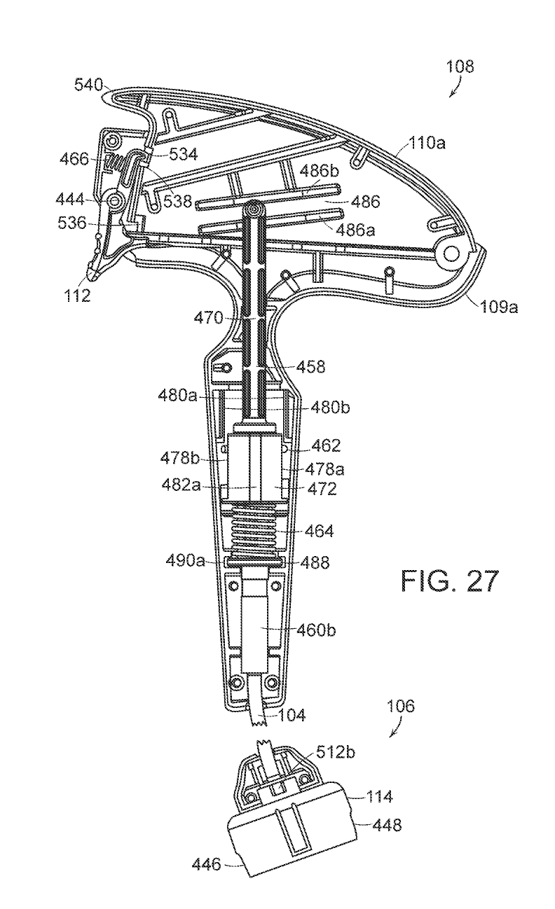

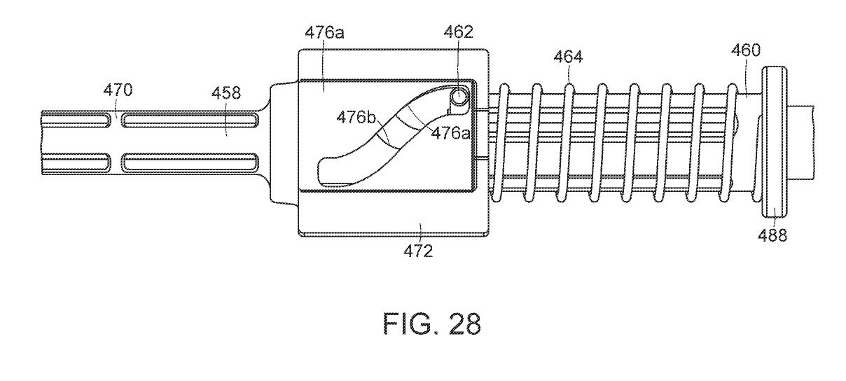

FIG. 26 is a side view of one embodiment of the deployment handle, applier, and flexible shaft shown in FIG. 24 with one of the two body halves omitted showing the internal components in the unapplied, non-actuated position. FIG. 27 is a side view of one embodiment of the deployment handle, applier, and flexible shaft shown in FIG. 24 with one of the two body halves omitted showing the internal components in the applied, actuated position. FIG. 28 is an enlarged fragmentary side view of one embodiment of a linear to rotary cam mechanism of the deployment handle shown in FIG. 24. As shown in FIGS. 25-28, the deployment handle 108 includes a cam 458, a drive shaft 460 to couple to the flexible shaft 104, a drive shaft pin 462, a cam return spring 464, a safety biasing spring 466, and an actuator mechanism 468 that couples to the fastener actuator 136. The actuator mechanism 468 is configured to effect the deployment or undeployment of the attachment mechanism of the camera 102 by rotating the fastener actuator 136. The cam 458 includes a shaft 470 and a cam collar 472. The upper end of the shaft 470 has a “T” configuration terminating in a cross member 474. The cam collar 472 defines a hollow interior and a pair of spaced apart, complementarily shaped cam tracks 476a, 476b (see FIG. 28) formed on opposite sides of the cam collar 472. Upper end 460a of the drive shaft 460 is disposed partially within the hollow interior defined by the cam collar 472, captured therein by the drive shaft pin 462. The drive shaft pin 462 is sized such that each end is located within the respective cam tracks 476a, 476b. The length of the hollow interior allows the upper end 460a to reciprocate therein, with the cam tracks 476a, 476b imparting rotation to the drive shaft 460 through the drive shaft pin 462 during reciprocation. The cam 458, the drive shaft 460, and the actuator mechanism 468 may be made of any suitable material having sufficient stiffness and strength. In the illustrated embodiment, the cam 458 and the actuator mechanism 468 are made of a liquid crystal polymer such as VECTRA™ LCP, and the drive shaft 460 is made of a PPE+PS such as NORYL™. The drive shaft pin 462 and the cam return spring 464 may be made of any suitable material, such as stainless steel.

The cam 458 is retained between the first and second body portions 109a, 109b, and in one embodiment, can reciprocate. The cam collar 472 has spaced apart, generally flat outer surfaces 478a, 478b tracks through which surfaces 476a, 476b are formed. The surfaces 476a, 476b are disposed between guide walls 480a, 480b formed in the first and second body portions 109a, 109b. The cam collar 472 also includes oppositely facing channels 482a, 482b (not illustrated), which are guided for axial reciprocation by the guides 484a, 484b (not illustrated) formed in the first and second body portions 109a and 109b, respectively. The upper end of the shaft 470 and the cross member 474 are disposed sandwiched between the first and second trigger halves 110a, 110b. Each of the first and second trigger halves 110a, 110b, includes a cam track 486 defined by a pair of spaced apart walls 486a, 486b extending from the interior surfaces of the first and second trigger the halves 110a, 110b. The cam track 486 is configured to receive and guide the cross member 474 as the trigger 110 is rotated about the pin 450, forcing the cam 458 to advance linearly downwardly into the body 109.

The drive shaft 460 includes an annular collar 488 which is received in slots 490a, 490b (not illustrated) formed in the respective first and second body halves 109a, 109b. The slots 490a, 490b rotatably support the drive shaft 460. The drive shaft 460 and the cam 458 are generally aligned and collinear with each other, defining the axis of the shaft portion of the body 109. As the cam 458 is advanced downwardly, the drive shaft pin 462 follows the cam tracks 476a and 476b, causing the drive shaft 460 to rotate, thus converting linear motion to rotary motion. The cam return spring 464 provides a nominal return force against the cam collar 472.

The shaft 104 is supported by a plurality of ribs 492, formed in each of the first and second body halves 109a, 109b that support the bend in the shaft 104 that permits the rotary motion to be transferred to the actuator mechanism 468, which may be disposed at an angle relative to the shaft of the body 109. The shaft 104 may be made of any suitable biocompatible material, such as stainless steel. In the illustrated embodiment, the shaft 104 has a stranded construction, with a center core having multiple layers of wire wrapped thereabout. First and second ends 104a, 104b of the shaft 104 may be attached to end 460b and the actuator mechanism 468, respectively, in any suitable manner which sufficiently limits rotational end play to prevent or minimize lost rotational motion. In the illustrated embodiment, the first end 104a of the shaft 104 is overmolded into the end 460b, and the second end 104b is press fit into the actuator mechanism 468. Alternatively, the first end 104a may be press fit into the end 460b, and the second end 104b may be overmolded into the actuator mechanism 468, both may be press fit, or both may be overmolded with a corresponding change to the configuration of the camera shroud 114 to allow assembly.

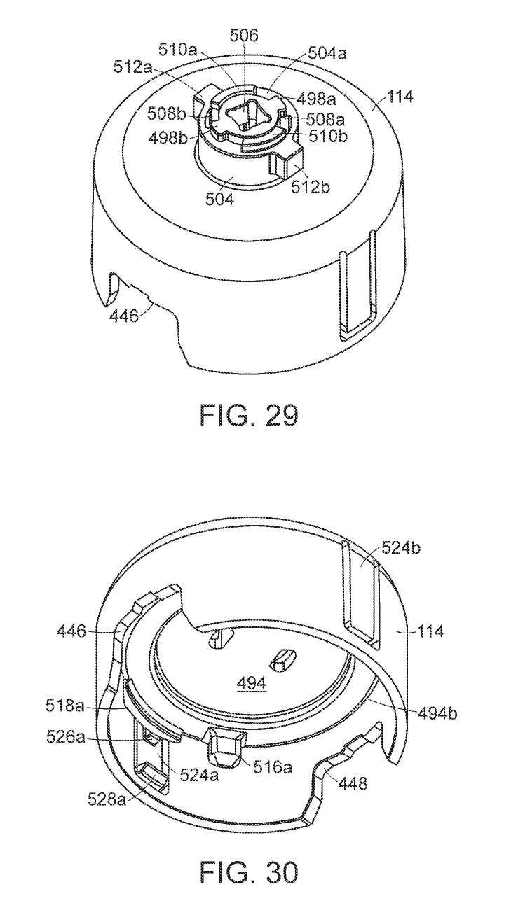

FIG. 29 is an enlarged top perspective view of one embodiment of a camera shroud of the applier shown in FIG. 24. As shown in FIGS. 25-29, the actuator mechanism 468 includes a disc shaped member 494 and a shaft 496 extending upwardly therefrom. The upper end of the shaft 496 includes a pair of outwardly extending tabs 498a, 498b. The camera shroud 114 includes a hub 504 defining a bore 506 therethrough. The bore 506 is shaped to receive and rotatably support the shaft 496 and includes two outwardly extending arcuate recesses 508a, 508b configured to provide assembly clearance for tabs 498a, 498b, allowing the hub 504 to be inserted into the bore 506. The lengths of the shaft 496 and the hub 504 are sized such that the tabs 498a, 498b are located above an upper surface 504a of the hub 504, allowing rotation of the actuator mechanism 468 while retaining it axially relative to the hub 504. Stops 510, 510b extend upwardly from upper the surface 504a, limiting the rotation of the actuator mechanism 468. The bore 506 defines a central axis of the camera shroud 114 about which the actuator mechanism 468 is rotated. The central axis of the camera shroud 114 is disposed at an angle to the axis of the shaft portion of the body 109, as previously mentioned. The hub 504 includes a pair of oppositely extending tabs 512a, 512b which retain the fastener actuator 136 to the body 109 and prevent rotation.

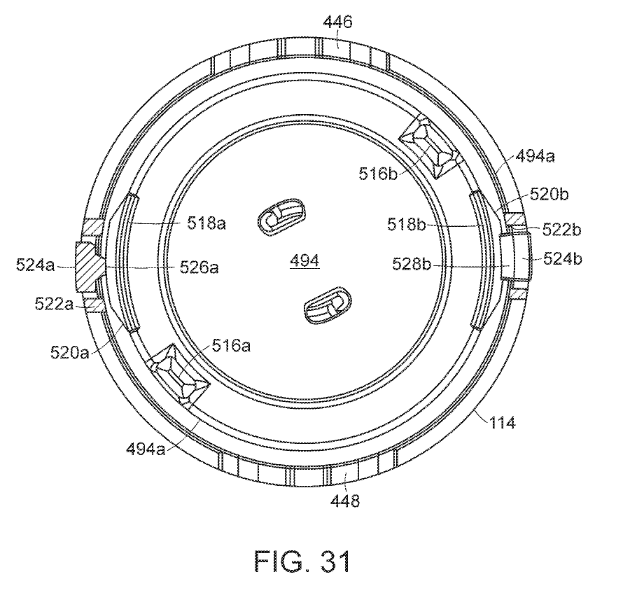

FIG. 30 is an enlarged bottom perspective view of one embodiment of a camera shroud and actuator portion of the applier shown in FIG. 24. FIG. 31 is a partial cutaway view end view of one embodiment of a camera shroud of the applier shown in FIG. 24. Referring to FIGS. 25-31, the disc shaped member 494 of the actuator mechanism 468 is shown disposed within the camera shroud 114. The actuator mechanism 468 includes a pair of spaced apart posts 516a, 516b, extending from adjacent periphery 494a of the member 494. The posts 516a, 516b are shaped complementarily with the openings 122. In the illustrated embodiment, the distal ends of posts the 516a, 516b are tapered to assist in guiding the posts 516a, 516b into the openings 122. Any suitable configuration may be utilized to create releasable contact between the actuator mechanism 468 and the fastener actuator 136 capable of actuating the fastener actuator 136.

The disc shaped member 494 also includes a pair of spaced apart cams 518a, 518b which extend outwardly and upwardly from a periphery 494a of the member 494. FIG. 31 is the cam 518a at a cross-section taken near the bottom surface of the member 494. The cams 518a, 518b include ramps 520a, 520b which start at a periphery 494a and lead out to respective surfaces 522a, 522b. Each surface 522a, 522b is arcuate, shown in the illustrated embodiment as generally having a constant radius.

In the illustrated embodiment, the camera shroud 114 includes a pair of the spaced apart cantilever arms 524a, 524b, each having ribs 528a, 528b, respectively. For clarity, FIG. 31 is the arm 524a in cross-section taken through the rib 528a, at the same level as for the cam 518a. At their distal ends, the cantilever arms 524a, 524b include respective inwardly extending flanges 528a, 528b. The flanges 528a, 528b are shaped complementarily to recesses 130a, 130b on the camera body 404, configured to engage the ledges 134a, 134b when the camera 102 is retained by the camera shroud 114.

In the illustrated embodiment, in the non-actuated state, the posts 516a, 516b are generally aligned with the cantilever arms 524a, 524b, respectively, although the posts 516a, 516b may be at any position that correspond to a position of the actuating feature of the actuator 136, which are depicted as openings 122a, 122b in the illustrated embodiment. As the trigger 110 is depressed, the actuator mechanism 468 rotates (counterclockwise in the illustrated embodiment when viewed from the bottom), advancing the cams 518a, 518b such that the ramps 520a, 520b contact the ribs 528a, 528b, respectively, deflecting the cantilever arms 524a, 524b outwardly. When the surfaces 522a, 522b engage the ribs 528a, 528b, the cantilever arms 524a, 524b are deflected a distance sufficient to move the flanges 528a, 528b to a position where they no longer extend into the recesses 116 or the contact ledges 130, thus releasing the camera 102 from the camera shroud 114.

FIG. 32 illustrates one embodiment of a temporarily positionable medical device comprising forward and rearward image acquisition capabilities. In one embodiment, the temporarily positionable medical device comprises an image acquisition system to provide visualization of the patient's anatomy in a rearward mode in the direction indicated by arrow “A” and in a forward mode in the direction indicated by arrow “B.” The forward viewing mode in the direction indicated by arrow “B” is employed during the delivery and deployment phase of the temporarily positionable medical device. The rearward viewing mode in the direction indicated by arrow “A” is employed to acquire images while the temporarily positionable medical device is attached to the patient's anatomy. In one embodiment, the temporarily positionable visualization system comprises a temporarily positionable device that may be deployed using minimally invasive surgical procedures (e.g., endoscopic, laparoscopic, thoracoscopic, or any combination thereof).

In the embodiment illustrated in FIG. 32, a camera applier system 600 comprises a temporarily positionable camera 602, a shaft 604, an applier 606, and a deployment handle 608. In one embodiment, the shaft 604 may be a flexible or articulating shaft or tube. The shaft 604 may be similar to the shaft 114 described above. Prior to deployment, the camera 602 is preloaded into the applier 606. The camera 602 comprises an image sensor 639 suitable for capturing light and converting optical images to electrical signals that can be stored in electronic storage media or transmitted to external devices for displaying the images in real-time. The electrical signals can be transmitted on a wire or wirelessly. Prior to intubating the camera 602 into an endoscopic trocar, the endoscopist (e.g., clinician, physician, or surgeon) attaches the applier 606 and the preloaded camera 602 to the shaft 604. The camera 602 is then deployed through the endoscopic trocar into a desired anatomical location inside the patient (e.g., deployment site) using fasteners integral with the camera 602. The embodiments, however, are not limited in this context as other techniques may be employed to deliver the camera 602 to the deployment site.

In one embodiment, the applier 606 is suitably configured to contain the camera 102 and is coupled to the deployment handle 608 via the shaft 604. The shaft 604 is flexible and suitable for deploying the applier 606 and the camera 602 via the inner working channel of a flexible endoscope, for example. The deployment handle 608 is coupled to the camera 602 via the applier 606. The camera 602 is preloaded into the applier 606 prior to deployment via the flexible endoscope through an endoscopic trocar. The camera 602 may comprise an attachment mechanism suitable for attaching the camera 602 to the desired tissue at a desired location.

In one embodiment, the camera 602 comprises a first lens 638a. The first lens 638a may be an optical lens or a system of lenses optically coupled to the image sensor 639 contained within a body 635 portion of the camera 602. The first lens 638a couples light to the image sensor 639 from a rearward direction indicated by arrow “A” when the camera 602 is deployed. The camera 602 also comprises one or more light sources 640a, 640b to illuminate the desired area to be imaged.

A suitably configured camera shroud 614 contains the camera 602 and provides for forward viewing in the direction indicated by arrow “B” during the delivery and deployment phase. In one embodiment, the camera shroud 614 comprises an optical channel 654, a two-way mirror 652, and a second lens 638b forming an illumination/optical path 650. The two-way mirror 652 may be a half-silvered mirror or a beam splitter to reflect some percentage of the light and pass some other percentage of the light. When the shroud 614 is coupled to the camera 602, light from the light source 640b is reflected by the two-way mirror 652 and directed through the optical path 654 to form an illumination beam 656 in the direction indicated by arrow “B” to illuminate the forward path during the delivery and deployment phase of the camera 602. Optical images 658 are reflected back to the optical path 654 through the second lens 638b and are directed to a portion of the image sensor 639 through the illumination/optical path 654. This provides a low-resolution image during the deployment phase of the camera 602. In one embodiment, the image sensor 639 comprises one or more arrays of CCDs or CMOS devices such as active-pixel sensors to capture light and convert the images 658 into electrical signals.

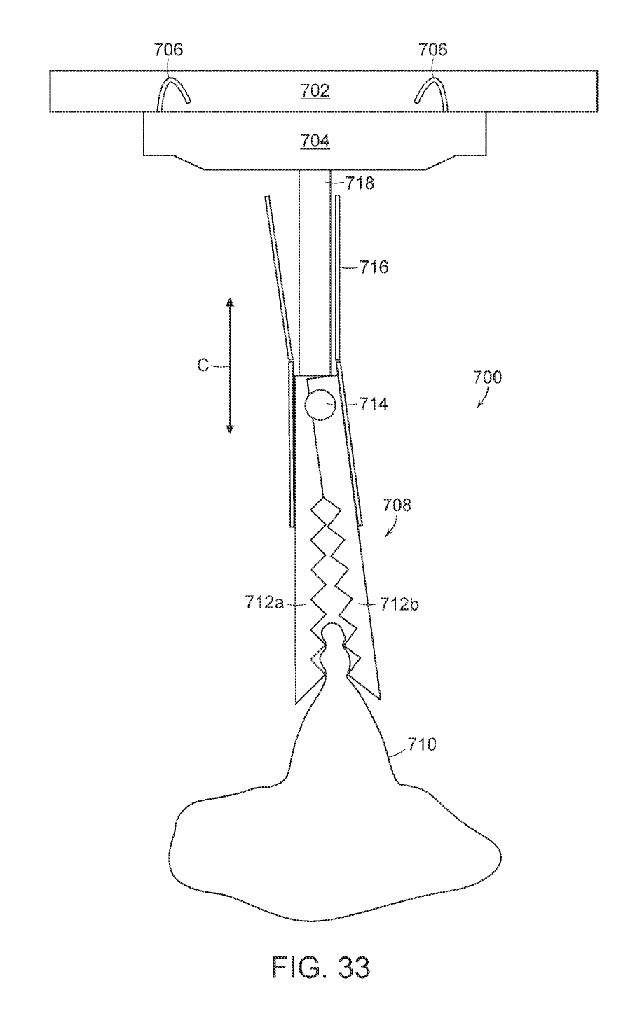

FIG. 33 illustrates one embodiment of a temporarily positionable medical device comprising a tissue retraction clip. In the illustrated embodiment, a temporarily positionable medical device 700 comprises an applier 704. A first end of the applier 704 comprises a support member 718 configured to engage a retraction clip 708. A second end of the applier 704 comprises one or more fasteners 706 to penetrate tissue 702 and attach the temporarily positionable medical device 700 thereto. In one embodiment, the fasteners 706 are similar to the fasteners 120 previously described and may be deployed to penetrate the tissue 702 in a similar manner. The retraction clip 708 may be employed to grasp tissue 710 between first and second jaws 712a, 712b pivotable about pivot point 714. The jaws 712a, 712b may be opened or closed by a sleeve 716 that is slidably movable is the direction indicated by arrow “C.”

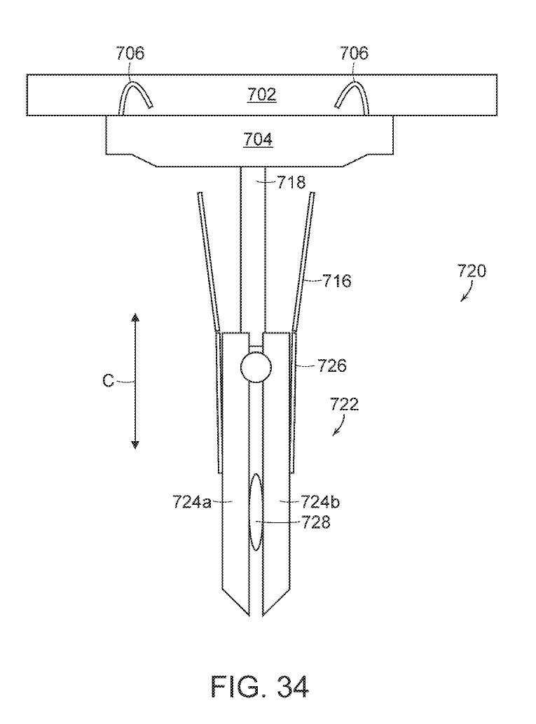

FIG. 34 illustrates one embodiment of a temporarily positionable medical device comprising a tissue clamp. In the illustrated embodiment, a temporarily positionable medical device 720 comprises an applier 704. A first end of the applier 704 comprises a support member 718 configured to engage a tissue clamp 722. A second of the applier 704 comprises one or more fasteners 706 to penetrate tissue 702 and attach the temporarily positionable medical device 720 thereto. The tissue clamp 722 may be employed to grasp tissue 728 between first and second jaws 724a, 724b pivotable about pivot point 726. The jaws 724a, 724b may be opened or closed by a sleeve 716 that is slidably movable is the direction indicated by arrow “C.”

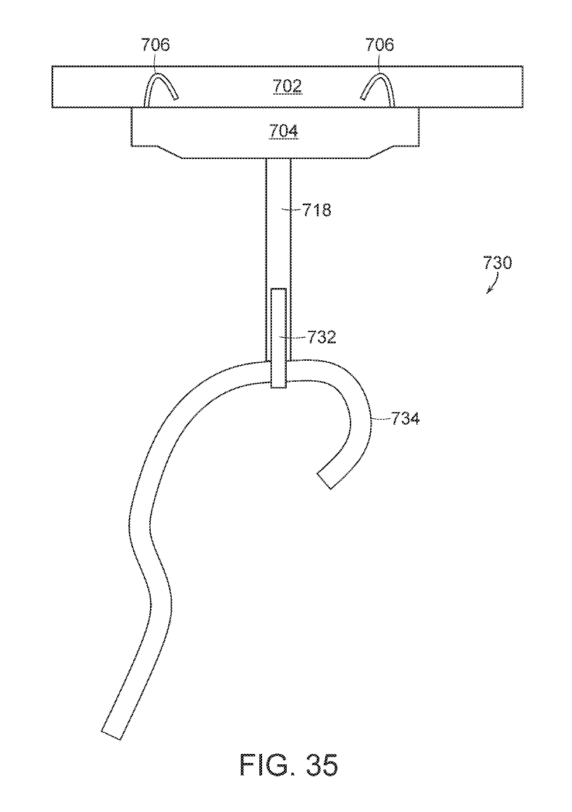

FIG. 35 illustrates one embodiment of a temporarily positionable medical device comprising a stabilizer clamp. In the illustrated embodiment, a temporarily positionable medical device 730 comprises an applier 704. A first end of the applier 704 comprises a support member 718 configured to engage a stabilizer clamp 734. A second end of the applier 704 comprises one or more fasteners 706 to penetrate tissue 702 and attach the stabilizer clamp 734 thereto. The stabilizer clamp 732 may be employed to grasp and stabilize a flexible portion of an endoscope 734.

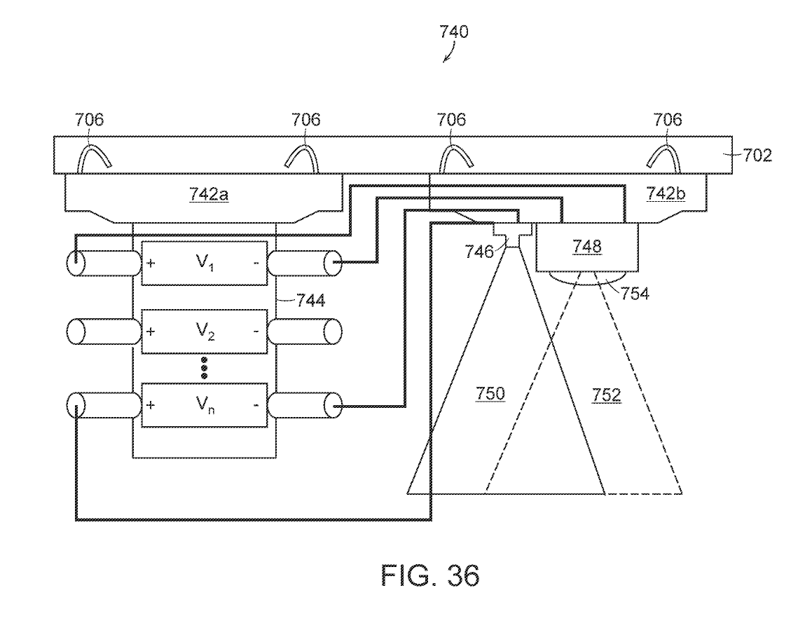

FIG. 36 illustrates one embodiment of a temporarily positionable medical device comprising an electrical power distributor, a light source, and a camera. In the illustrated embodiment, a first applier 742a comprises an electrical power distributor 744. A first end of the first applier 742a is configured to support the electrical power distributor 744 and a second end is configured to penetrate tissue 702 with one or more fasteners 706 to attach the electrical power distributor 744 to the tissue 702. A second applier 742b comprises the light source 746 and the camera 748. A first end of the second applier 742b is configured to support the illumination device 746 and the camera 748 and a second end is configured to penetrate tissue 702 with one or more fasteners 706 to attach the light source 746 and the camera 748 to the tissue 702.

The electrical power distributor 744 comprises one or more voltage sources V₁, V₂, Vₙ, where n is any positive integer. The voltage sources V₁, V₂, Vₙ may be electrically coupled to the light source 746, the camera 748, or any other electrical device. The voltage sources V₁−Vₙ may supply any suitable voltage. In one embodiment, the voltage source V₁ may supply about +12V to power the camera 748 and the voltage source Vₙ may supply about +1.5V to power the light source 746. V₂ may supply about +5V to power other devices. In one embodiment, the illumination device 746 may be an LED. The light source 746 generates light 750 to illuminate the target anatomical area. The camera lens 754 receives light 752 reflected from the illuminated target anatomical area.

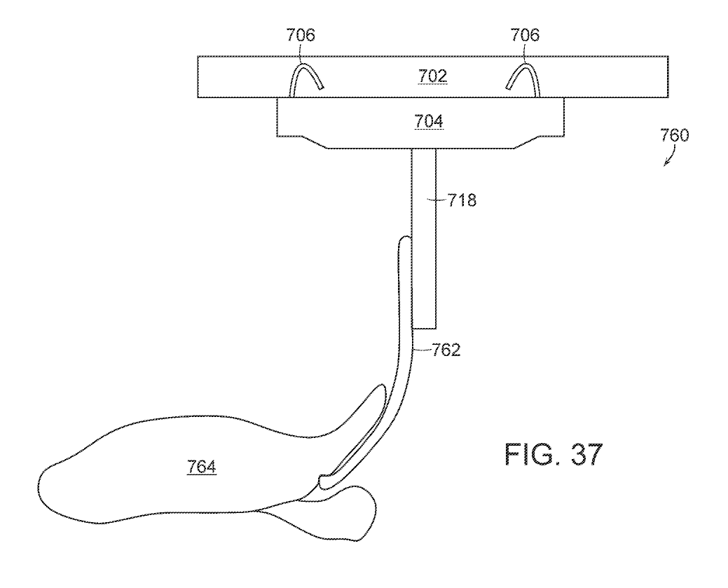

FIG. 37 illustrates one embodiment of a temporarily positionable medical device comprising a tissue spreader to create space between layers of tissue. In the illustrated embodiment, a temporarily positionable medical device 760 comprises an applier 704. A first end of the applier 704 comprises a support member 718 configured to attach to a tissue spreader 762. A second end of the applier 704 comprises one or more fasteners 706 to penetrate tissue 702 to attach the tissue spreader 762 thereto. The tissue spreader 762 may be employed to separate layers of tissue, for example.

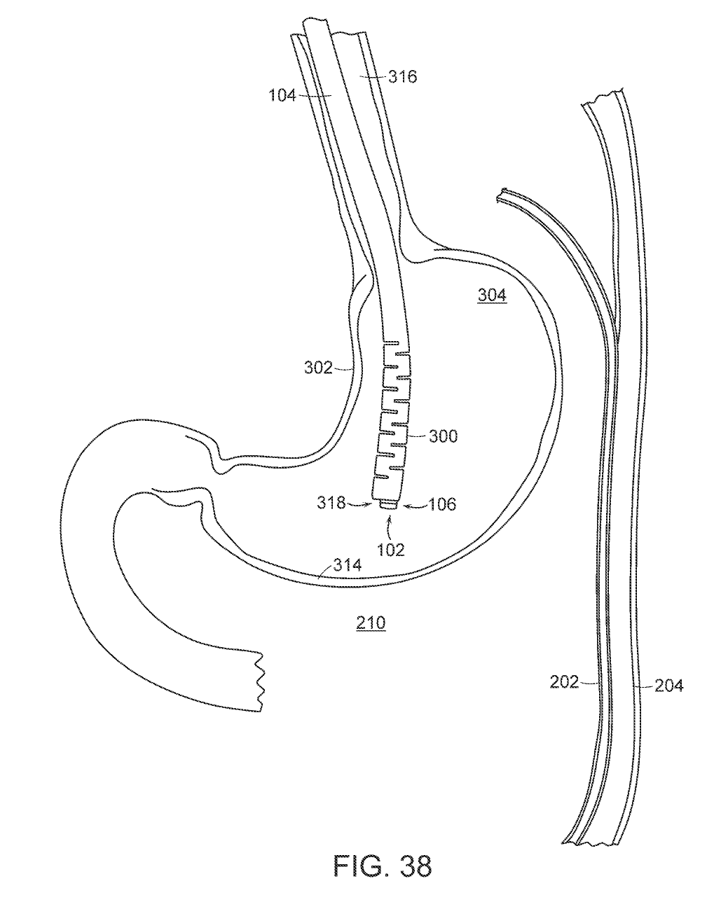

FIGS. 38-43 illustrate a procedure for deploying a temporarily positionable medical device into the abdominal wall 202 in a natural orifice translumenal endoscopic surgical procedure. Although the procedure is described in the context of the camera 102, the same procedure may be employed to deploy other temporarily positionable medical device disclosed herein or temporarily positionable medical devices that fall within the scope of the embodiments disclosed herein. For example, the same procedure may be used to deploy the camera 105 or various types of temporarily positionable end-effectors introduced through the flexible working channel of an endoscope including, but not limited to retraction clips, tissue clamps, endoscope stabilizers, electrical power distribution devices, and/or devices to create space between internal body lumen, organs, and/or dissected sections of tissue. Prior to deployment, the camera 102 is locked into the applier 106 and is intubated into the stomach cavity 304 using an endoscopic trocar 300 guided through the gastrointestinal tract 316. The abdominal cavity 210 is then accessed through the stomach 302 by inserting an endoscopic veress needle (EVN) access device 306 through the stomach wall 314. A hole 312 is formed by the penetrating access device 306 and is dilated using a balloon 308. The applier 106 and the endoscopic trocar 300 are pushed through the dilated hole 312 in the stomach wall 314 into the abdominal cavity 210. When the distal end 318 of the endoscopic trocar 300 is close to the abdominal wall 202, the shaft 104 is extended until the applier 106 is in contact with the abdominal wall 202. Then, the camera 102 is deployed and attached to the abdominal wall 202 using the one or more fasteners 120 and the shaft 104 is retracted through the stomach 302 and the gastrointestinal tract 316. The camera 102 deployment process is described in more detail below. In one embodiment, the endoscopic trocar 300 may be guided using visualization feedback from the camera 105 comprising the forward viewing optics.

FIG. 38 is a cross-sectional view of a stomach cavity, gastrointestinal tract, and abdominal wall showing an endoscopic trocar intubated within the stomach cavity through the gastrointestinal tract. In the illustrated embodiment, the endoscopic trocar 300 is intubated within the stomach cavity 304 through the gastrointestinal tract 316. The applier 106 is visible at the distal end 318 of the endoscopic trocar 300 within the stomach cavity 304. The camera 102 is preloaded into the applier 106 and is configured to be attached to the abdominal wall 202. The endoscopic trocar 300, the applier 106, and the camera 102 are initially introduced into the stomach 302. The endoscopic trocar 300, the applier 106, and the camera 102 may be introduced through the stomach 302 to a tissue treatment region within the abdominal cavity 210 using well known endoscopic transgastric access methods.

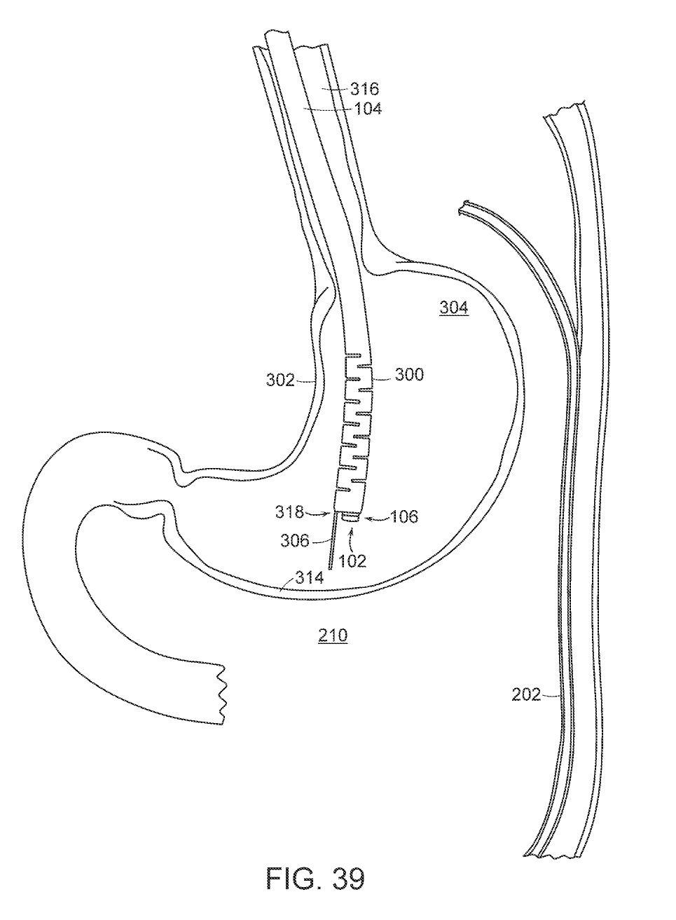

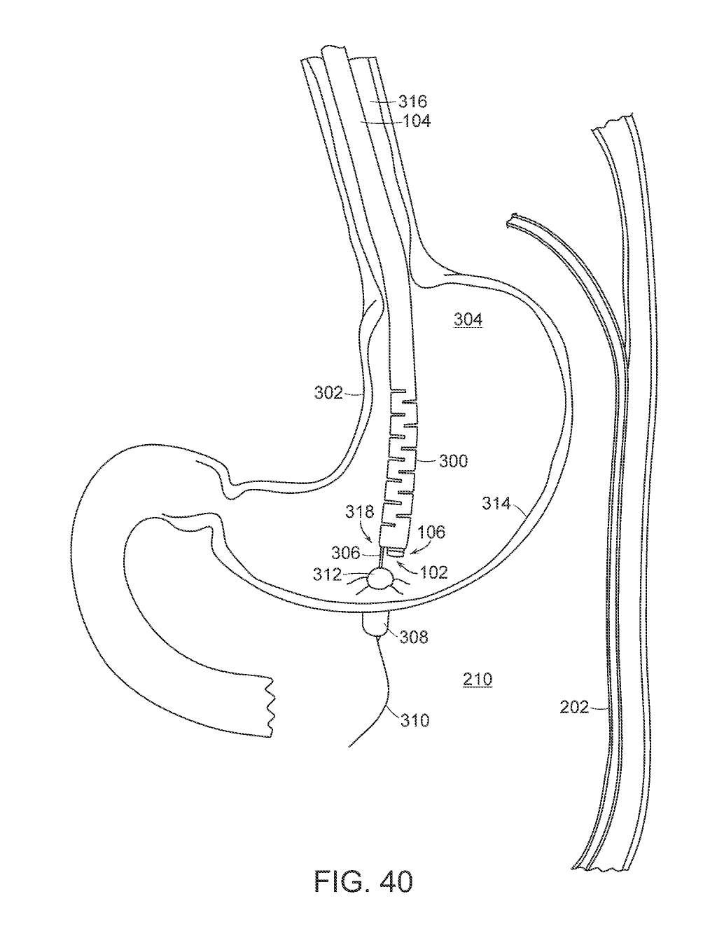

FIG. 39 is a cross-sectional view of the stomach cavity, gastrointestinal tract, and abdominal wall shown in FIG. 38 showing an access device extending from the distal end of the endoscopic trocar. Once the endoscopic trocar 300 is intubated within the stomach cavity 304 through the gastrointestinal tract 316, the access device 306 is extended through the distal end 318 of the endoscopic trocar 300. The access device 306 is inserted through the working channel of a flexible endoscope introduced through the endoscopic trocar 300. The access device 306 is used to penetrate the stomach wall 314 to access the abdominal cavity 210 and perform a medical procedure at the worksite within the abdominal cavity 210.

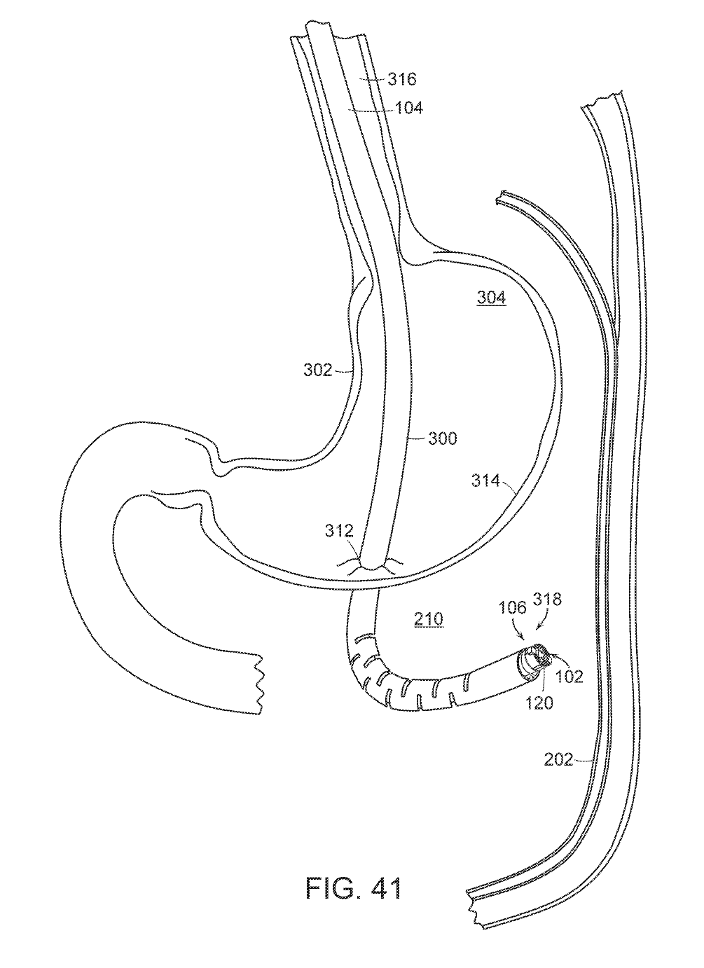

FIG. 40 is a cross-sectional view of the stomach cavity, gastrointestinal tract, and abdominal wall shown in FIG. 39 showing a dilating balloon inserted through an opening formed in the stomach wall by the access device 306. Once the access device 306 penetrates the stomach wall 314 and forms a small opening 312 in the stomach wall 314, a balloon 308 is inserted through the opening 312 and is inflated to dilate the opening 312. The opening 312 may be sufficiently dilated to enable the endoscopic trocar 300 to pass therethrough. A guide wire 310 is attached to the balloon 308 to pull the balloon 308 through the dilated opening 312 and into the abdominal cavity 210. The endoscopic trocar 300 and the applier 106 are then pushed through the dilated opening 312 into the abdominal cavity 210.

FIG. 41 is a cross-sectional view of a stomach cavity, gastrointestinal tract, and abdominal wall shown in FIG. 40 showing a distal end of an endoscopic trocar intubated inserted through the dilated opening formed in the stomach wall. As shown in FIG. 41, the distal end 318 of the endoscopic trocar 300 is introduced into the abdominal cavity 210 through the dilated opening 312. The endoscopic trocar 300, the applier 106, and the camera 102 are advanced within the abdominal cavity 210 towards the abdominal wall 202. The distal end 318 of the endoscopic trocar 300 is guided towards the abdominal wall 202 until it reaches a point where the shaft 104 can be extended outwardly towards the abdominal wall 202.

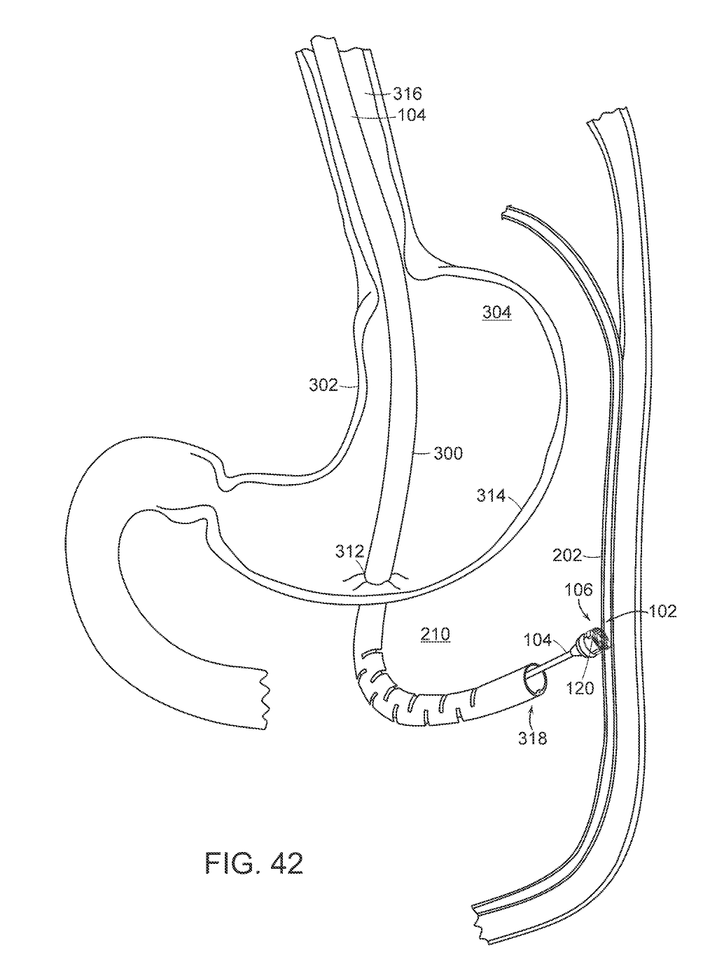

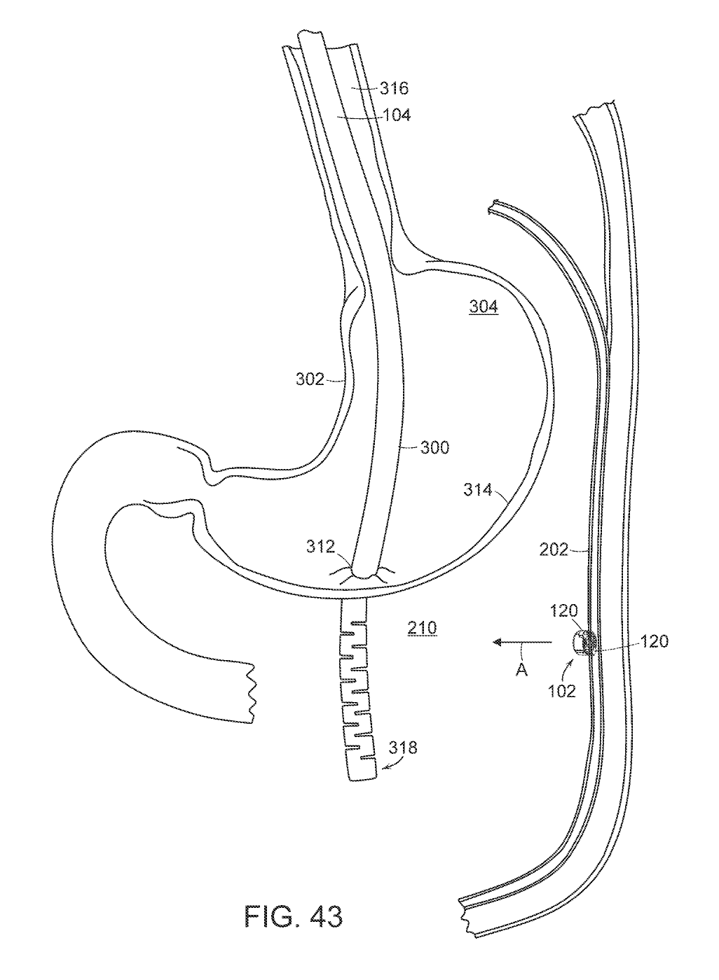

FIG. 42 is a cross-sectional view of the stomach cavity, gastrointestinal tract, and abdominal wall shown in FIG. 41 showing the flexible shaft and the applier extended through the distal end of the endoscopic trocar. As shown in FIG. 42, the shaft 104 is advanced trough the distal end 318 of the endoscopic trocar 300 and is extended beyond the distal end 318 of the endoscopic trocar 300 until the applier 316 is in contact with the abdominal wall 202. Once the applier 106 and the camera 102 are placed in contact with the abdominal wall 202, the deployment handle 108 is actuated to deploy the fasteners 120 and attach the camera 102 to the abdominal wall 202.

FIG. 43 is a cross-sectional view of the stomach cavity, gastrointestinal tract, and abdominal wall shown in FIG. 42 showing one embodiment of a temporarily positionable medical device attached to the abdominal wall. In the illustrated embodiment, the camera 102 is deployed and attached to the abdominal wall 202. With the camera 102 in position, the one or more fasteners 120 are moved from the undeployed position to the deployed position in an annular path to engage the tissue. The fasteners 120 allow the camera 102 to be secured to the tissue with retention strength equal to or greater than that achievable with sutures. Once the fasteners 120 are deployed into the abdominal wall 202 tissue, the fasteners 120 attach the camera 102 thereto. Once the camera 102 is attached to the abdominal wall 202, the shaft 104 is retracted through the abdominal cavity 210, the opening 312, the stomach 302, and an upper gastrointestinal tract 316 of the patient. Once the camera 102 is attached to the abdominal wall 202, the camera 102 provides the desired surgical view of the anatomy of the abdominal cavity 210.

With reference now also to FIGS. 25-31, the attachment mechanism is configured to be reversible so that the temporarily positionable medical device, e.g., the camera 102, may be moved, such as to reposition it or remove it from the patient. To do so, with the trigger 110 in the deployed position, the camera shroud 114 is placed over the camera 102 and the outwardly extending portions 124a, 124b are located in the respective slots 446, 448 so that the posts 516a, 516b are engaged with the recesses 122. The safety switch 112 is rotated to withdraw the lockout tab 534 from the upper opening 538, while the clinician pulls up on the extension 540 of the trigger 110. Although the cam return spring 464 urges the cam collar 472 upwardly, the extension 540 allows an additional return force to be applied. As the cross member 474 is pulled up by the cam track 486, the actuator mechanism 468 rotates the actuator 136, moving the fasteners 120 from the deployed position to the undeployed position simultaneously, while the cams 518a, 518b disengage from the ribs 528a, 528b, allowing the flanges 188a, 188b to engage the recess 130 and the ledge 130a so as to retain the camera 102 in the camera shroud 114. When the trigger 110 has been moved to the undeployed position, the lockout tab 534 snaps into the lower opening 536, generating an audible signal that the trigger 110 is undeployed fully, and the camera 102 is detached from the body tissue and may be relocated or removed.

In summary, numerous benefits have been described which result from employing the concepts described herein. The foregoing description of the one or more embodiments has been presented for purposes of illustration and description. It is not intended to be exhaustive or limiting to the precise form disclosed. Modifications or variations are possible in light of the above teachings. The one or more embodiments were chosen and described in order to illustrate principles and practical application to thereby enable one of ordinary skill in the art to utilize the various embodiments and with various modifications as are suited to the particular use contemplated. It is intended that the claims submitted herewith define the overall scope.

The temporarily positionable devices disclosed herein can be designed to be disposed of after a single use, or they can be designed to be used multiple times. In either case, however, the temporarily positionable devices can be reconditioned for reuse after at least one use. Reconditioning can include any combination of the steps of disassembly of the temporarily positionable device, followed by cleaning or replacement of particular pieces, and subsequent reassembly. In particular, the temporarily positionable device can be disassembled, and any number of the particular pieces or parts of the temporarily positionable device can be selectively replaced or removed in any combination. Upon cleaning and/or replacement of particular parts, the temporarily positionable device can be reassembled for subsequent use either at a reconditioning facility, or by a surgical team immediately prior to a surgical procedure. Those skilled in the art will appreciate that reconditioning of a temporarily positionable device can utilize a variety of techniques for disassembly, cleaning/replacement, and reassembly. Use of such techniques, and the resulting reconditioned temporarily positionable device, are all within the scope of the present application.

Preferably, the various embodiments described herein will be processed before surgery. First, a new or used temporarily positionable device is obtained and if necessary cleaned. The temporarily positionable device can then be sterilized. In one sterilization technique, the temporarily positionable device is placed in a closed and sealed container, such as a plastic or TYVEK® bag. The container and the temporarily positionable device are then placed in a field of radiation that can penetrate the container, such as x-rays, or high-energy electrons. The radiation kills bacteria on the instrument and in the container. Other sterilization techniques, such as Ethylene Oxide (EtO) gas sterilization also may be employed to sterilize the temporarily positionable device prior to use. The sterilized temporarily positionable device can then be stored in the sterile container. The sealed container keeps the temporarily positionable device sterile until it is opened in the medical facility.

It is preferred that the temporarily positionable device is sterilized. This can be done by any number of ways known to those skilled in the art including beta or gamma radiation, ethylene oxide, steam.

Although various embodiments have been described herein, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A temporarily positionable device comprising:
a temporarily positionable body;
an attachment mechanism formed integral with the temporarily positionable body to attach to body tissue, the attachment mechanism comprising at least one fastener integral with the temporarily positionable body to attach the temporarily positionable body to the body tissue, at least one fastener has a deployed position and an undeployed position; and
an applier to move at least one fastener from the undeployed position to the deployed position.

2. The device of claim 1, comprising:
a trigger; and
a lockout button coupled to the trigger;
wherein the applier is to move the at least one fastener from the undeployed position to the deployed position by the actuating the trigger in a first direction; and
wherein the applier is to move the at least one fastener form the deployed position to the undeployed position by engaging the lockout button and actuating the trigger.

3. The device of claim 1, comprising a plurality of fasteners.

4. The device of claim 1, comprising a camera disposed in the temporarily positionable body;

5. The device of claim 4, wherein the camera comprises at least one lens.

6. The device of claim 5, comprising a first image sensor optically coupled to the at least one lens.

7. The device of claim 5, wherein the camera comprises a second lens.

8. The device of claim 5, comprising a second image sensor optically coupled to the second lens.

9. The device of claim 1, comprising a tissue retraction clip disposed in the temporarily positionable body.

10. The device of claim 1, comprising a tissue clamp disposed in the temporarily positionable body.

11. The device of claim 1, comprising an endoscope stabilizer disposed in the temporarily positionable body.

12. The device of claim 1, comprising an electrical power distributor disposed in the temporarily positionable body.

13. The device of claim 1, comprising a temporary space creation device disposed in the temporarily positionable body.

14. The device of claim 1, comprising first and second power input terminals.

15. The device of claim 14, wherein the first and second power input terminals are adapted to couple to corresponding first and second input terminals of a battery.

16. The device of claim 14, wherein the first and second power input terminals are adapted to couple to corresponding first and second percutaneous needle electrodes to couple the camera to a power source external to the body tissue.

17. The device of claim 14, wherein the first and second power input terminals are adapted to couple to corresponding first and second flexible conductors to couple the camera to a power source external to the body tissue via a path through a natural orifice of the patient.

18. The device of claim 1, comprising at least one percutaneously located conductor to transmit captured images to an external monitor.

19. The device of claim 1, comprising a flexible conductor that egresses the patient via a natural orifice of the patient to transmit captured images to an external monitor.

20. The device of claim 1, comprising a radio frequency (RF) component coupled to the camera to wirelessly transmit images to the external monitor using RF energy.

21. The device of claim 1, comprising an actuator to move the fastener from the undeployed position to the deployed position.

22. The device of claim 21, wherein the actuator is to move the fastener from the undeployed position to the deployed position.

23. The device of claim 21, wherein the actuator is configured to resist an undeploying force applied to the fastener when the fastener is disposed in the deployed position.

24. The device of claim 23, wherein the undeploying force is a rotational force.

25. The device of claim 21, wherein the fastener is configured to rotate about a respective axis as it moves from the undeployed position to the deployed position.

26. The device of claim 25, wherein the actuator is configured to rotate about a respective axis, and comprises an associated surface for the fastener, the surface is configured to exert a rotational force on the fastener when the actuator is rotated in a deploying direction to move the fastener from the undeployed position to the deployed position.

27. The device of claim 25, wherein the actuator is configured to resist being rotated by an undeploying rotational force applied to the fastener when the fastener is disposed in the deployed position.

28. The device of claim 27, wherein the attachment mechanism comprises a detent system configured to resist unintended rotation of the actuator.

29. The device of claim 25, wherein the actuator is configured to rotate about a respective axis, and the attachment mechanism comprises an associated member for the fastener, the member being carried by the actuator, the actuator, the fastener and its associated member being configured such that the member exerts a rotational force on each associated fastener when the actuator is rotated in an undeploying direction to move the fastener from the deployed position to the undeployed position.

30. The device of claim 29, wherein the fastener and its associated member are configured such that the member does not exert a deploying force when the actuator is rotated in a deploying direction.

31. The device of claim 21, wherein the actuator is rotatable.

32. The device of claim 31, wherein the actuator comprises a generally annular ring which is rotatably carried by the temporarily positionable body.

33. The device of claim 21, wherein the actuator is configured to be manipulated to move the fastener by any of one or more standard surgical instruments.

34. The device of claim 21, wherein the fastener comprises a distal tip configured to pierce body tissue, the fastener being disposed in an associated recess of the temporarily positionable body, the distal tip being disposed in the associated recess when the fastener is disposed in the undeployed position.

35. The device of claim 34, wherein the fastener is disposed completely within the associated recess when the fastener is disposed in the undeployed position.

36. The device of claim 34, wherein the distal tip is disposed in an associated recess, the distal tip being disposed in the associated recess when the fastener is disposed in the deployed position.

37. The device of claim 21, wherein the fastener comprises a distal tip configured to pierce body tissue, the fastener being disposed in an associated recess, the distal tip being disposed in the associated recess when the fastener is disposed in the deployed position.

38. A method of surgically positioning a temporarily positionable medical device, the method comprising:
disposing a temporarily positionable medical device and a fastener integral to the temporarily positionable medical device at a first location adjacent to body tissue; and
simultaneously moving the fastener from an undeployed position to a deployed position to attach the temporarily positionable medical device to the body tissue at the first location.

39. The method of claim 38, comprising the moving the fastener from the deployed position to the undeployed position to detach the temporarily positionable medical device from the body tissue.

40. The method of claim 38, comprising:
guiding the temporarily positionable medical device to the first location by viewing images from a camera contained within the temporarily positionable medical device.

41. The method of claim 40, comprising:
transmitting images from the camera while guiding the temporarily positionable medical device to the first location.

42. The method of claim 38, comprising:
disposing the temporarily positionable medical device at a second location adjacent body tissue; and simultaneously moving the fastener from the undeployed position to the deployed position to attach the temporarily positionable medical device to the body tissue at the second location.

43. The method of claim 38, comprising disposing the temporarily positionable medical device and the fastener integral to the temporarily positionable medical device through a flexible portion of a flexible trocar.

44. A method comprising:
obtaining a temporarily positionable device comprising:
a temporarily positionable body;
an attachment mechanism formed integral with the temporarily positionable body to attach the camera to a body tissue, the attachment mechanism comprising a fastener integral to the temporarily positionable body to attach the temporarily positionable body to tissue, the fastener has a deployed position and an undeployed position; and
an applier to move the fastener from the undeployed position to the deployed position;
sterilizing the temporarily positionable device; and
storing the temporarily positionable device in a sterile container.
